(19) United States
(12) Patent Application Publication
(10) Pub. No.: US 2019/0345246 A1
(43) Pub. Date: Nov. 14, 2019

(54) Compositions and Methods for Treating Autoimmune Inner Ear Disease

(71) Applicant: TympoBio, Inc., Morrisville, NC (US)

(72) Inventor: Thomas Jung, Zug (CH)

(21) Appl. No.: 16/407,868

(22) Filed: May 9, 2019

Related U.S. Application Data
(60) Provisional application No. 62/669,415, filed on May 10, 2018.

(51) Int. Cl. C07K 16/24; A61P 27/16; A61K 9/00

(52) U.S. Cl. CPC C07K 16/245; A61K 9/0029; A61P 27/16

(57) ABSTRACT

The invention relates to methods for treating autoimmune inner ear disease (AIED). In particular, the invention relates to treating AIED with humanized anti-IL-1β antibodies or fragments thereof, especially monovalent, highly potent anti-IL-1β antibody fragments. The invention further relates to antibodies, compositions and kits for use in the methods of the invention.

### CROSS-REFERENCE TO RELATED APPLICATIONS

This application claims the benefit of U.S. Provisional Application No. 62/669,415, filed May 10, 2018, the entire contents of which are incorporated by reference herein in its entirety.

### FIELD OF THE INVENTION

The invention relates to antibodies, compositions, and methods for treating autoimmune inner ear disease (AIED). In particular, the invention relates to treating AIED with humanized anti-IL-1β antibodies, especially monovalent, highly potent anti-IL-1β antibody fragments.

### BACKGROUND OF THE INVENTION

Autoimmune inner ear disease (AIED) is a syndrome of progressive hearing loss that is caused by antibodies or immune cells attacking the inner ear. In most cases, there is a reduction of hearing accompanied by tinnitus (ringing, hissing, roaring) which typically occurs in one ear before spreading to the other ear. Less than about 1% of the 28 million Americans suffering from hearing loss are diagnosed with AIED and about 30% of people diagnosed with AIED are already being treated for an autoimmune disease affecting their whole bodies (such as rheumatoid arthritis, lupus, scleroderma, ulcerative colitis, or Sjoegren's Syndrome).

The causes of AIED are not well understood, but there are several theories based on the knowledge obtained from related immune disorders: (a) Bystander damage-damage to the inner ear causes cytokines to be released which provoke (after a delay) additional immune reactions; (b) Cross reacting-antibodies or T-cells cause accidental inner ear damage because the ear shares common antigens with a potentially harmful substance, virus, bacteria that the body is fighting off, e.g., CTL2 has recently been reported as a potential target antigen in AIED (Kommareddi et al 2009); (c) Intolerance—the inner ear may be only a partially immune privileged locus. This means that the body may not recognize all of the inner ear antigens, and when they are released (perhaps following surgery or an infection), the body may wrongly amount an attack on the “foreign” antigen; and (d) Genetic factors-genetically controlled aspects of the immune system may increase or otherwise be associated with increased susceptibility to common hearing disorders.

Diagnosis of AIED is often difficult because the symptoms are initially often mistaken for an ear infection. There is no direct test for diagnosing AIED and blood tests for conditions resembling autoimmune disorders are typically performed when screening for AIED Current treatment options for AIED primarily comprise regimens of steroids or chemotherapy type medication. The initial responsiveness to these therapies supports the hypothesis of AIED as an immune-mediated disorder. High doses of steroids, such as prednisone and dexamethasone, are initially prescribed to reduce inflammation but can only be used for several weeks due to the adverse side effects associated with long term usage of high doses of steroids. Therefore, chemotherapy type medications such as methotrexate and/or Cytoxan are often prescribed for long term use, which exhibit less undesirable side effects. Ongoing efforts to identify better treatment options for AIED include studies with immunosuppressive drugs such as the T-cell drug ORENCIA® or anti-TNF alpha drug ENBREL® as well as evaluating cell/gene therapy treatments. However, results of these studies are very preliminary and, thus, there still remains a great need in the art to develop better treatment options for AIED

### SUMMARY OF THE INVENTION

The invention is based, in part, on methods of treating autoimmune inner ear disease (AIED) in a subject in need thereof comprising delivering to the subject a therapeutically effective amount of an antibody that specifically binds to interleukin-1β (IL-1β), thereby treating AIED. IL-1β has been identified as a key mediator in the inflammatory cascade and therefore has been associated with numerous autoimmune diseases comprising an inflammatory component. AIED is an autoimmune disease with an inflammatory component, and thus the current disclosure is directed toward treating AIED with an IL-1β antibody. The antibody can be a full length immunoglobulin or a fragment thereof, such as a bivalent fragment F(ab)₂, such as scFv. In some embodiments, antibody binding to IL-1β primarily exhibit an IC₅₀ of 30 pM or less. In some embodiments, the antibody structurally comprises:
a. at least one of the variable heavy chain (VH) CDR sequences CDR-H1, CDR-H2 or CDR-H3 as set forth in SEQ ID NOS: 1, 2 and 3, respectively, or variants thereof, and/or
b. at least one of the variable light chain (VL) CDR sequences CDR-L1, CDR-L2 or CDR-L3 as set forth in SEQ ID NOS: 4, 5, and 6, respectively, or variants thereof.

In one embodiment, the antibody comprises the amino acid sequence of SEQ ID NO: 10. The antibody may be formulated into a pharmaceutical composition, such as for intratympanic (e.g., into the middle ear), subcutaneous, and/or transdermal (e.g., a patch) administration. Administration of the antibody may be performed every 1, 2, 3, 4, 5 or 6 months to improve symptoms such as inflammation, hearing loss, dizziness, vertigo, tinnitus and/or fullness of the ear. The subject may be a human and may already be on a regimen of anti-inflammatories and/or immunosuppressive agents for treating AIED or an already existing immune disorder.

### BRIEF DESCRIPTION OF THE DRAWINGS

FIG. 1 shows neutralization of human IL-1β with DLX-2323.

FIG. 2 shows a graphical representation of scFv versus IgG concentrations (ng/mL) in RHE medium determined at time point t=48 hours.

FIG. 3 shows inhibition of hIL-1β induced systemic IL-6 in mouse.

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 2nd Ed. (Cold Spring Harbor, N.Y., 1989); Ausubel et al. Current Protocols in Molecular Biology (Green Publishing Associates, Inc. and John Wiley & Sons, Inc., New York).

### I. Definitions

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of an antibody, compound or agent of this invention, dose, time, temperature, and the like, is meant to encompass variations of ±10%, ±5%, ±1%, ±0.5%, or even ±0.1% of the specified amount.

The term “consisting essentially of” (and grammatical variants), as applied to an amino and/or nucleotide sequence of this invention, means an amino and/or nucleotide sequence that consists of both the recited sequence (e.g., SEQ ID NO) and a total of ten or less (e.g., 1, 2, 3, 4, 5, 6, 7, 8, 9, or 10) additional amino acids and/or nucleotides on the N-terminal end and/or C-terminal end and/or 5′ and/or 3′ ends of the recited sequence such that the ability of the an amino and/or nucleotide sequence to bind to its target is not materially altered. For example, the total of ten or less additional nucleotides includes the total number of additional nucleotides on both the 5′ and 3′ ends added together. The term “materially altered,” as applied to the binding of the nucleotide sequence, refers to an increase or decrease in binding affinity of at least about 50% or more as compared to the binding affinity of a nucleotide sequence consisting of the recited sequence.

Within the scope of the present invention, the term “antibody” refers to full-length immunoglobulins as well as to fragments thereof. Such full-length immunoglobulins may be monoclonal, polyclonal, chimeric, humanized, veneered or human antibodies.

The term “antibody fragments” comprises portions of a full-length immunoglobulin retaining the targeting specificity of said immunoglobulin. Many but not all antibody fragments lack at least partially the constant region (Fc region) of the full-length immunoglobulin. In some embodiments, antibody fragments are produced by digestion of the full-length immunoglobulin. An antibody fragment may also be a synthetic or recombinant construct comprising parts of the immunoglobulin or immunoglobulin chains (see e.g., HOLLIGER, P. and Hudson, J. Engineered antibody fragments and the rise of single domains. Nature Biotechnology 2005, vol. 23, no. 9, p. 1126-1136). Examples of antibody fragments, without being limited to, include scFv, Fab, Fv, Fab′, F(ab′)₂ fragments, dAb, VHH, nanobodies, V(NAR) or minimal recognition units. “Single chain variable fragments” or “single chain antibodies” or “scFv” are one type of antibody fragments. scFv are fusion proteins comprising the VH and VL of immunoglobulins connected by a linker. They thus lack the constant Fc region present in full-length immunoglobulins, but retain the specificity of the original immunoglobulin.

The “IC₅₀” or “half-maximum inhibitory concentration” is a measure of antagonist drug potency and describes quantitatively the effectiveness of a compound to inhibit a biological or biochemical function. This measure indicates how much of the compound is needed to inhibit by 50% a certain biological or biochemical process. Although no direct indicator of affinity, both values are correlated and can be determined via the Cheng-Prusoff equation (CHENG Y. and Prusoff W. H. Relationship between the inhibition constant (Ki) and the concentration of inhibitor which causes 50 percent inhibition (ISO) of an enzymatic reaction. Biochemical Pharmacology 1973, vol. 22, p. 3099-3108; RAMMES, G., et al. Identification of a domain which affects kinetics and antagonistic potency of clozapine at 5-HT3 receptors. PLOS one 2009, vol. 4, p. 1-14; ZHEN, J., et al. Concentration of receptor and ligand revisited in a modified receptor binding protocol for high-affinity radioligands: [³H] spiperone binding to D₂ and D₃ dopamine receptors. Journal of Neuroscience Methods 2010, vol. 188, p. 32-38).

The term “IL-1β specific binding” as used herein describes that a binding member binds to IL-1β with higher affinity than to a structurally different antigen which does not comprise the IL-1β epitope to which the anti-IL-1β binding member binds. Specific binding is reflected by a dissociation equilibrium constant (K_{D}) of lower than 1 micromolar. This constant can be determined, e.g., using Quartz Crystal Microbalance (QCM) in an Attana instrument, or Surface Plasmon Resonance (SPR) technology in a BIACORE instrument.

As used herein, “IL-1β” refers to the molecule as described in, e.g., Dinarello C. A., Treating inflammation by blocking interleukin-1 in a broad spectrum of diseases. Nature reviews 2012, vol. 11, p. 633-652. “hIL-1β” as used herein refers to human IL-1β. “rIL-1β” refers to recombinant IL-1β. Recombinant IL-1β may or may not have an amino terminal methionine residue, depending upon the method by which it is prepared. “rhIL-1β” refers to recombinant human IL-1β. rhIL-1β may, e.g., be obtained from Peprotech, USA, cat. no. 200-01B. IL-1β may also be obtained by isolation from biological samples of human or non-human origin.

As used herein “humanized” antibodies refer to antibodies comprising one or more, typically all six CDR regions of a non-human parent antibody or variants thereof, and of which the framework is, e.g., (i) a human framework, potentially comprising one or more framework residues of the non-human parent antibody, or (ii) a framework from a non-human antibody modified to increase similarity to naturally produced human frameworks. Methods of humanizing antibodies are known in the art, see e.g. LEGER, O. and Saldanha, J. Antibody Drug Discovery. Edited by WOOD, C. London: Imperial College Press, 2011. ISBN 1848166281. p. 1-23.

As used herein “framework” (FR) refers to the scaffold of the variable immunoglobulin domain, either the variable light chain (VL) or variable heavy chain (VH), embedding the respective CDRs. A VL and/or VH framework typically comprises four framework sections, FR1, FR2, FR3 and FR4, flanking the CDR regions. Thus, as known in the art, a VL has the general structure: (FR-L1)-(CDR-L1)-(FR-L2)-(CDR-L2)-(FR-L3)-(CDR-L3)-(FR-L4), whereas a VH has the general structure: (FR-H1)-(CDR-H1)-(FR-H2)-(CDR-H2)-(FR-H3)-(CDR-H3)-(FR-H4).

As used herein “CDR” refers to the hypervariable regions of the antibody which mainly contribute to antigen binding. Typically, an antigen binding site comprises six CDRs, embedded into a framework scaffold. Herein, the CDRs of the VL are referred to as CDR-L1, CDR-L2 and CDR-L3 whereas the CDRs of the VH are referred to as CDR-H1, CDR-H2 and CDR-H3. These can be identified as described in KABAT, E. A., et al. Sequences of Proteins of Immunological Interest. 5th edition. Edited by U.S. DEPARTMENT OF HEALTH AND HUMAN SERVICES. NIH Publications, 1991. p. 91-3242. CDR-H1 as used herein, however, differs from the Kabat definition in that it starts with position 27 and ends prior to position 36.

As used herein, the numbering system to identify amino acid residue positions in the VH and VL of the antibody corresponds to the “AHo”-system described by HONEGGER, A. and Plückthun, A. Yet another numbering scheme for immunoglobulin variable domains: An automatic modelling and analysis tool. Journal of Molecular Biology 2001, vol. 309, p. 657-670. The publication further provides conversion tables between the AHo and the Kabat system (KABAT, E. A., et al. Sequences of Proteins of Immunological Interest. 5th edition. Edited by U.S. DEPARTMENT OF HEALTH AND HUMAN SERVICES. NIH Publications, 1991. p. 91-3242).

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain.

The term “identity” as used herein refers to the sequence match between two proteins or nucleic acids. The protein or nucleic acid sequences to be compared are aligned to give maximum identity, for example using bioinformatics tools such as EMBOSS Needle (pair wise alignment; available at www.ebi.ac.uk). When the same position in the sequences to be compared is occupied by the same nucleobase or amino acid residue, then the respective molecules are identical at that very position. Accordingly, the “percent identity” is a function of the number of matching positions divided by the number of positions compared and multiplied by 100%. For instance, if 6 out of 10 sequence positions are identical, then the identity is 60%. The percent identity between two protein sequences can, e.g., be determined using the Needleman and Wunsch algorithm (NEEDLEMAN, S. B. and Wunsch, C. D. A general method applicable to the search for similarities in the amino acid sequence of two proteins. Journal of Molecular Biology 1970, vol. 48, p. 443-453) which has been incorporated into EMBOSS Needle, using a BLOSUM62 matrix, a “gap open penalty” of 10, a “gap extend penalty” of 0.5, a false “end gap penalty”, an “end gap open penalty” of 10 and an “end gap extend penalty” of 0.5. Two molecules having the same primary amino acid or nucleic acid sequence are identical irrespective of any chemical and/or biological modification. For example, two antibodies having the same primary amino acid sequence but different glycosylation patterns are identical by this definition. In case of nucleic acids, for example, two molecules having the same sequence but different linkage components such as thiophosphate instead of phosphate are identical by this definition.

As used herein, the term “similar” refers to protein sequences which, when aligned, share similar amino acid residues and most often, but not mandatorily, identical amino acid residues at the same positions of the sequences to be compared. Similar amino acid residues are grouped by chemical characteristics of the side chains into families. Said families are described below for “conservative amino acid substitutions.” The “percent similarity” between sequences is the number of positions that contain identical or similar residues at the same sequence positions of the sequences to be compared divided by the total number of positions compared and multiplied by 100%. For instance, if 6 out of 10 sequence positions have identical amino acid residues and 2 out of 10 positions contain similar residues, then the sequences have 80% similarity. The similarities between two sequences can, e.g., be determined using EMBOSS Needle.

As used herein, the term “variant” refers to an amino acid or nucleic acid sequence which differs from the parental sequence by virtue of addition (including insertions), deletion and/or substitution of one or more amino acid residues or nucleobases while retaining at least one desired activity of the parent sequence disclosed herein. In the case of antibodies such desired activity may include specific antigen binding. Similarly, a variant nucleic acid sequence may be modified when compared to the parent sequence by virtue of addition, deletion and/or substitution of one or more nucleobases, but the encoded antibody retains the desired activity as described above. Variants may be naturally occurring, such as allelic or splice variants, or may be artificially constructed.

As used herein, the term “conservative modifications” refers to modifications that are physically, biologically, chemically or functionally similar to the corresponding reference, e.g., has a similar size, shape, electric charge, chemical properties, including the ability to form covalent or hydrogen bonds, or the like. Such conservative modifications include, but are not limited to, one or more nucleobases and amino acid substitutions, additions and deletions.

For example, conservative amino acid substitutions include those in which the amino acid residue is replaced with an amino acid residue having a similar side chain. For example, amino acid residues being non-essential with regard to binding to an antigen can be replaced with another amino acid residue from the same side chain family, e.g., serine may be substituted for threonine. Amino acid residues are usually divided into families based on common, similar side-chain properties, such as:

1. nonpolar side chains (e.g., glycine, alanine, valine, leucine, isoleucine, methionine),
2. uncharged polar side chains (e.g., asparagine, glutamine, serine, threonine, tyrosine, proline, cysteine, tryptophan),
3. basic side chains (e.g., lysine, arginine, histidine, proline),
4. acidic side chains (e.g., aspartic acid, glutamic acid),
5. beta-branched side chains (e.g., threonine, valine, isoleucine) and
6. aromatic side chains (e.g., tyrosine, phenylalanine, tryptophan, histidine).

A conservative substitution may also involve the use of a non-natural amino acid. Non-conservative substitutions, i.e., exchanging members of one family against members of another family, may lead to substantial changes, e.g., with respect to the charge, dipole moment, size, hydrophilicity, hydrophobicity or conformation of the binding member, which may lead to a significant drop in the binding activity, in particular if amino acids are affected that are essential for binding to the target molecule. A non-conservative substitution may also involve the use of a non-natural amino acid.

Conservative and non-conservative modifications can be introduced into parental binding members by a variety of standard techniques known in the art, such as combinatorial chemistry, site-directed DNA mutagenesis, PCR-mediated and/or cassette mutagenesis, peptide/protein chemical synthesis, or chemical reaction specifically modifying reactive groups in the parental binding member. The variants can be tested by routine methods for their chemical, biological, biophysical and/or biochemical properties.

Nucleic acid hybridization reactions can be performed under conditions of different stringency. “Stringent conditions” are widely known and published in the art. Typically, during the hybridization reaction a SSC-based buffer can be used in which SSC is 0.15 M NaCl and 15 mM citrate buffer having a pH of 7.0. Increasing buffer concentrations and the presence of a denaturing agent increase the stringency of the hybridization step. For example, high stringency hybridization conditions can involve the use of: (i) 50% (vol/vol) formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C. with washes at 42° C. in 0.2×SSC and 0.1% SDS; (ii) 50% (vol/vol) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (iii) 10% dextran sulfate, 2×SSC, and 50% formamide at 55° C., followed by a high-stringency wash consisting of 0.1×SSC containing EDTA at 55° C. Additionally or alternatively, one, two or more washing steps using wash solutions of low ionic strength and high temperature can be included in the hybridization protocol using, for example, 0.015 M sodium chloride/0.0015 M sodium citrate/0.1% sodium dodecyl sulfate at 50° C.

The term “autoimmune disorders,” as used herein, refers to any disorder associated with an autoimmune reaction. Examples include, without limitation, multiple sclerosis, Crohn's disease, ulcerative colitis, lupus, psoriasis and rheumatoid arthritis.

By the terms “treat,” “treating,” or “treatment of,” it is intended that the severity of the subject's condition is reduced or at least partially improved or modified and that some alleviation, mitigation or decrease in at least one clinical symptom is achieved.

An “effective” amount as used herein is an amount that provides a desired effect.

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject (e.g., in the case of autoimmune inner ear disease, reduction in inflammation and/or hearing loss). Those skilled in the art will appreciate that the therapeutic effects need not be complete or curative, as long as some benefit is provided to the subject.

The term “dispersing agents,” and/or “viscosity modulating agents” are materials that control the diffusion and homogeneity of the antibody disclosed herein through liquid media. Examples of diffusion facilitators/dispersing agents include but are not limited to hydrophilic polymers, electrolytes, Tween® 60 or 80, PEG, polyvinylpyrrolidone (PVP; commercially known as Plasdone), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., PLURONICS F68®, F88®, and F108®, which are block copolymers of ethylene oxide and propylene oxide); and poloxamines (e.g., TETRONIC 908®, also known as POLOXAMINE 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol has a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose are also be used as dispersing agents. Dispersing agents useful in liposomal dispersions and self-emulsifying dispersions of the antibody disclosed herein are dimyristoyl phosphatidyl choline, natural phosphatidyl choline from eggs, natural phosphatidyl glycerol from eggs, cholesterol and isopropyl myristate.

The terms “enhance” or “enhancing” refers to an increase or prolongation of either the potency or duration of a desired effect of the antibody, or a diminution of any adverse symptomatology that is consequent upon the administration of the therapeutic agent. Thus, in regard to enhancing the effect of the antibody disclosed herein, the term “enhancing” refers to the ability to increase or prolong, either in potency or duration, the effect of other therapeutic agents that are used in combination with the antibody disclosed herein. An “enhancing-effective amount,” as used herein, refers to an amount of antibody or other therapeutic agent which is adequate to enhance the effect of another therapeutic agent or antibody of the target auris structure in a desired system. When used in a patient, amounts effective for this use will depend on the severity and course of AIED, previous therapy, the patient's health status and response to the drugs, and the judgment of the treating physician.

As used herein, a “pharmaceutical device” includes any composition described herein that, upon administration, provides a reservoir for extended release of an active agent (i.e., antibody) described herein.

The term “solubilizers” refer to auris-acceptable compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like that assist or increase the solubility of the antibody disclosed herein.

The term “stabilizers” refers to compounds such as any antioxidation agents, buffers, acids, preservatives and the like which are compatible with the environment of the auris interna. Stabilizers include but are not limited to agents that will do any of (1) improve the compatibility of excipients with a container, or a delivery system, including a syringe or a glass bottle, (2) improve the stability of a component of the composition, or (3) improve formulation stability.

The term “diluent” refers to chemical compounds that are used to dilute the antimicrobial agent prior to delivery and which are compatible with the auris internal.

The term “surfactants” refer to compounds that are auris-acceptable, such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., PLURONIC® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants are included to enhance physical stability or for other purposes.

By “auris-pharmaceutically acceptable,” as used herein, refers to a material, such as a carrier or diluent, which does not abrogate the biological activity or properties of the compound in reference to the auris interna (or inner ear), and is relatively or is reduced in toxicity to the auris interna (or inner ear), i.e., the material is administered to an individual without causing undesirable biological effects or interacting in a deleterious manner with any of the components of the composition in which it is contained.

The term “auris interna” refers to the inner ear, including the cochlea and the vestibular labyrinth, and the round window that connects the cochlea with the middle ear.

The term “auris media” refers to the middle ear, including the tympanic cavity, auditory ossicles and oval window, which connects the middle ear with the inner ear.

The term “carrier materials” are excipients that are compatible with the active agent, the auris interna and the release profile properties of the auris-acceptable pharmaceutical formulations. Such carrier materials include, e.g., binders, suspending agents, disintegration agents, filling agents, surfactants, solubilizers, stabilizers, lubricants, wetting agents, diluents, and the like.

The term “auris-pharmaceutically compatible carrier materials” include, but are not limited to, acacia, gelatin, colloidal silicon dioxide, calcium glycerophosphate, calcium lactate, maltodextrin, glycerine, magnesium silicate, polyvinylpyrrolidone (PVP), cholesterol, cholesterol esters, sodium caseinate, soy lecithin, taurocholic acid, phosphatidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like.

### II. Therapeutic Applications

As one aspect, the invention provides methods of treating autoimmune inner ear disease (AIED) in a subject in need thereof by delivering to the subject a therapeutically effective amount of an antibody that specifically binds to IL-1β, thereby treating AIED The antibody of the invention is either a full length immunoglobulin or an antibody fragment (e.g., a Fab, Fab′, F(ab)2, or scFv).

In some embodiments, the antibody of the invention is administered so that the antibody is in contact with the crista fenestrae cochleae, the round window membrane or the tympanic cavity. In one embodiment, the antibody is administered locally into the ear canal or in the vestibule of the ear. Access to, for example, the vestibular and cochlear apparatus will occur through the auris media including the round window membrane, the oval window/stapes footplate, the annular ligament and through the otic capsule/temporal bone. In one embodiment, the antibody is administered by intratympanic injection, e.g., on or near the round window membrane, or into the middle ear.

Intratympanic injection of therapeutic agents is the technique of injecting a therapeutic agent behind the tympanic membrane into the middle and/or inner ear. In one embodiment, the antibody described herein is administered directly onto the round window membrane via transtympanic injection. In another embodiment, the antibody described herein is administered onto the round window membrane via a non-transtympanic approach to the inner ear. In additional embodiments, the antibody described herein is administered onto the round window membrane via a surgical approach to the round window membrane comprising modification of the crista fenestrae cochleae. With this route of administration the disclosed antibody can be delivered directly to the site of treatment (i.e., the inner ear) compared to administering the antibody systemically. In some embodiments, the concentration of the antibody at the treatment site is higher when administered locally (e.g., intratympanic/transtympanic injection) compared to the concentration of the antibody at the treatment site when administered systemically.

In one embodiment, the delivery system for administering the antibody of the invention is a syringe and needle apparatus that is capable of piercing the tympanic membrane and directly accessing the round window membrane or crista fenestrae cochleae of the auris interna. In some embodiments, the needle on the syringe is wider than an 18 gauge needle. In another embodiment, the needle gauge is from 18 gauge to 31 gauge. In a further embodiment, the needle gauge is from 25 gauge to 30 gauge. The gauge level of the syringe or hypodermic needle can vary. In another embodiment, the internal diameter of the needle can be increased by reducing the wall thickness of the needle (commonly referred as thin wall or extra thin wall needles) to reduce the possibility of needle clogging while maintaining an adequate needle gauge. In another embodiment, the needle is a hypodermic needle used for instant delivery of the antibody.

In some embodiments, the delivery device is an apparatus designed for administration of therapeutic agents to the middle and/or inner ear. By way of example only: GYRUS Medical Gmbh offers micro-otoscopes for visualization of and drug delivery to the round window niche; Arenberg has described a medical treatment device to deliver fluids to inner ear structures in U.S. Pat. Nos. 5,421,818; 5,474,529; and 5,476,446, each of which is incorporated by reference herein for such disclosure. U.S. Pat. No. 6,045,528, which is incorporated herein by reference for such disclosure, describes a surgical method for implanting a fluid transfer conduit to deliver therapeutic agents to the inner ear. U.S. Patent Application Publication 2007/0167918, which is incorporated herein by reference for such disclosure, further describes a combined otic aspirator and medication dispenser for intratympanic fluid sampling and medicament application.

In some embodiments, the antibody of the invention is administered parenterally. Examples of parenteral administration include, but should not be limited to, subcutaneous administration, intradermal administration, intravenous administration, intramuscular administration, and transdermal administration. In some embodiments, the antibody of the invention is administered subcutaneously. Subcutaneous administration delivers the antibody of the invention into a tissue layer between the skin and the muscle as a bolus injection. The volume of the bolus injection can vary. In some embodiments, the volume ranges from about 0.1 mL to about 2.0 mL, from about 0.1 mL to about 1.5 mL, from about 0.1 mL to about 1.0 mL, or from about 0.1 mL to about 0.5 mL. The site of administration can vary. In some embodiments, the antibody of the invention is administered subcutaneously to body parts such as, but not limited to, the side and/or back of the upper arm, abdomen, front of thigh, upper back, and/or upper area of the buttocks. After subcutaneous administration the antibody of the invention absorbs into the bloodstream at a constant rate until a therapeutically effective concentration of the antibody is reached and maintained over a time period to treat AIED The treatment period can last for hours, days, weeks and/or months. With subcutaneous administration the antibody of the invention is not immediately exposed to the liver and/or GI tract of the subject thereby avoiding first pass metabolism.

In one embodiment, the delivery system of administering the antibody of the invention is a syringe and needle apparatus that is capable of injecting the antibody into the subcutis, which is the layer of the skin below the dermis and the epidermis. The gauge of the needle can vary. In some embodiments, the needle on the syringe is from 25 gauge to 30 gauge or from 25 gauge to 27 gauge. The length of the needle can vary. In some embodiments, the length of the needle is from about ⅜ inch to about ⅝ inch. In some embodiments, the delivery system is an implantable device. In some embodiments, the device is a subdermal implant, wherein the device is implanted in a manner as to be completely buried in the dermis. In some embodiments, the device is a transdermal implant, wherein the device is partially placed under the skin with a portion of the device exposed.

In some embodiments, the antibody of the invention is administered transdermally. During transdermal administration the antibody of the invention is delivered across the skin into the bloodstream for systemic distribution. Examples for such a delivery device is a transdermal patch which typically comprises one or more of the following components: an adhesive to adhere the components of the patch together along with adhering the patch to the skin; a membrane to control the release of the antibody from the reservoir and multi-layer patches (which are both described in more detail below); a backing to protect the patch from the outer environment; a permeation enhancer to promote permeation of the antibody through the skin; a matrix filler to provide bulk to the matrix as well as to provide some fillers that act as a matrix stiffening agent; and an antibody of the invention. In some embodiments, the transdermal patch is a single-layer drug-in-adhesive patch, wherein the adhesive layer of the transdermal patch also contains the drug (i.e., antibody of the invention). In this type of patch the adhesive layer is surrounded by a temporary liner and a backing and serves two purposes: (a) adhering the various layers of the patch together; and (b) ensuring attachment of the patch to the skin so that the drug can be released from the patch into the skin. In some embodiments, the transdermal patch is a multi-layer drug-in adhesive patch, wherein another layer of drug-in-adhesive is added onto a single-layer drug-in adhesive patch using a membrane to separate the two layers. The first layer is for immediate release of the drug, while the other layer is for controlled release of the drug from the reservoir. The drug release can be controlled with the permeability properties of the membrane and the diffusion characteristics of the drug. In some embodiments, the transdermal patch is a reservoir patch, comprising a separate drug layer from the adhesive layer. The drug layer is a liquid compartment containing drug solution or suspension separated by the adhesive layer. The drug reservoir is encapsulated in a shallow compartment molded from a drug-impermeable material (e.g., a metallic plastic laminate with a rate-controlling membrane made of a polymer like material (e.g., vinyl acetate) on one surface). In some embodiments, the transdermal patch is a matrix patch, wherein the drug layer comprises a semisolid matrix containing a drug solution and/or suspension. The adhesive layer in this patch surrounds the drug layer, partially overlaying it. In some embodiments, the antibody of the invention comprised in the transdermal patch is able to transfer from the patch into the skin passing through the various layers of the skin (e.g., epidermis and dermis) to reach the microcirculation of the dermis. The microcirculation of the dermis contains small vessels that distribute the drug into the systemic circulation. The amount of the antibody of the invention comprised in the transdermal patch can vary and depends, in part, on the efficiency of the antibody of the invention to pass through the skin and the amount of antibody required for obtaining a therapeutic effect. The time required for the antibody to be released from the patch and travel through the skin can vary. In some embodiments, the time can take from one to several minutes to several hours, days, and/or weeks. Transdermal administration allows for the drug (i.e., antibody of the invention) to enter the systemic circulation without being exposed to first pass metabolism of the liver.

In some embodiments, the transdermal patch is a microneedle patch comprising an array of solid or hollow microneedles on a backing with an adhesive. Solid microneedles of microneedle patches are coated with and/or incorporate a drug (e.g., antibody), wherein the drug loading per needle can vary. For example, in some embodiments, the drug loading per needle can range up to a maximum amount of about 300 micrograms, about 200 micrograms, about 100 micrograms, about 75 micrograms, about 50 micrograms, about 25 micrograms, or about 10 micrograms. Microneedle patches with hollow microneedles delivery drug solutions (i.e., liquid formulations that are not particularly viscous) into the highly vascularized dermal layer of the skin. In some embodiments, an array of hollow needles of a patch is able to deliver up to a maximum of about 2 mL, about 1.5 mL, about 1.0 mL, about 0.75 mL, about 0.5 mL, or about 0.25 mL of drug formulation. The viscosity of the drug formulation can vary and is typically depended on the physical properties of the drug being formulated and the desired release characteristics of the transdermal patch, e.g., the amount of drug formulation released in a given time frame. The viscosity of the drug formulation is also suitable as to function properly with the device, for example, the viscosity is suitable as to not clog up the hollow needles in the patch.

During application, the microneedles are inserted into the skin to penetrate the epidermis and creating direct access to the dermis (i.e., intradermal administration). The microneedles can be made from any suitable material for this application such as medical grade polymers. Other suitable routes to deliver the antibody of the invention also include, without limitation, intrathecally, intraperitoneally, intrarectally, intravaginally, intranasally, intragastrically, intratracheally, or intrapulmonarily.

The amount of antibody administered to a subject is a therapeutically effective amount, which refers to an amount that is sufficient to improve at least one symptom, or otherwise hinder, retard or reverse the progression of AIED. For example, a therapeutically effective amount for treating AIED would be an amount that would improve at least one symptom such as hearing loss, dizziness, fullness in the ear, tinnitus, vertigo, or combinations thereof. In some embodiments, a therapeutically effective amount of the antibody is administered until such symptoms improve by at least 10%, 20%, 30%, 40%, 50%, 60%, or 70%. In other examples, a therapeutically effective amount of the antibody is administered to reduce inflammation in the inner ear. In some embodiments, a therapeutically effective amount of the antibody is administered until inflammation is reduced by at least 10%, 20%, 30%, 40%, 50%, 60%, or 70%.

As AIED is a chronic condition repeated administration of the disclosed antibody may be required. In some embodiments, the antibody is administered 1, 2, 3, 4, or 5 times a week. In some embodiments, the antibody is administered every 1, 2, 3, or 4 weeks. In some embodiments, the antibody is administered every 1, 2, 3, 4, 5, or 6 months. In some embodiments, the antibody is administered until inflammation, hearing loss, dizziness, fullness in the year, tinnitus, vertigo, or combinations thereof improve by at least 10%, 20%, 30%, 40%, 50%, 60%, or 70%. The frequency of administration may be altered over time, e.g., more frequently at first until therapy is achieved, and then less frequently to maintain the therapeutic effect.

In some embodiments, the antibody is administered in combination with at least one or more additional therapeutic agents, such as an anti-inflammatory agent and/or an immunosuppressive agent. The therapeutic agent can be administered simultaneously with the antibody or at different time points, wherein the route of administration can be the same or different as the route of administration of the antibody. In some embodiments, the therapeutic agent is formulated in the same pharmaceutical composition as the antibody. Exemplary anti-inflammatories include, but should not be limited to, COX-2 inhibitors, JAK inhibitors, leukotriene modifiers, corticosteroids, prostaglandin inhibitors, or NSAIDS. Exemplary immunosuppressive agents include, but should not be limited to, glucocorticoids, cytostatics, alkylating agents, antimetabolites, cytotoxic antibiotics, T-cell receptor directed monoclonal antibodies, IL-2 receptor directed antibodies, interferons, or macrolide lactones.

The subject in need of such treatment can be a human or a non-human animal, e.g., a mouse, rat, rabbit, monkey, dog, horse, cow, chicken, guinea pig or pig. Typically, the subject is a human. In some embodiments, the subject is already receiving treatment for AIED and/or a systemic autoimmune disorder. Examples of systemic autoimmune disorders include, but should not be limited to, multiple sclerosis, Crohn's disease, systemic lupus, inflammatory bowel syndrome, rheumatoid arthritis, psoriasis, vitiligo, and ulcerative colitis. In some embodiments, the subject is resistant to steroid treatment for AIED or has become resistant to steroid treatment for AIED after originally being sensitive to the treatment.

### III. Antibodies and Compositions

In a first aspect, the invention provides an antibody or fragment thereof binding IL-1β. In some embodiments, the antibody or fragment thereof competes for binding to IL-1β, e.g., human IL-1β, cynomolgus IL-1β, rhesus monkey IL-1β, and/or rat IL-1β, e.g., hIL-1β. Such antibodies may also include antibodies disclosed in U.S. Patent Publication No. 2016/0194392, which is incorporated by reference in its entirety. In some embodiments, the antibody or fragment thereof inhibits the biological effects of human IL-1β best with an IC₅₀ of lower than about 500, 300, 200, 100 or 50 pM. In some embodiments, said IC₅₀ is lower than 40, 30, 20, 10, 5, 4, 3, 2, or 1 pM. The IC₅₀ can, e.g., be determined using a cell based potency assay. In one embodiment, the IC₅₀ value is determined by inhibiting the IL-1β induced release of IL-6 from human fibroblasts. Such assay is based on the observation that fibroblasts stimulated with IL-1β release IL-6. In the presence of IL-1β inhibiting antibodies, the concentration of released IL-6 is reduced. In some embodiments, Normal Human Fibroblasts (NHDF-Neo, e.g., obtainable from Lonza Walkersville USA, cat, no CC-2509) cells are used. Upon incubation with a mixture of hIL-1β and the antibody of interest, supernatants are harvested and examined by IL-6 ELISA such as the R&D Systems Human IL-6 Duo Set ELISA kit (R&D Systems, cat. No. DY206). The IC₅₀ value may be the mean value obtained of at least three independent repetitions of such assay. In some embodiments, the antibody of fragment thereof retains specific binding to IL-1β, particularly to hIL-1β.

In some embodiments, there is no residual activity of IL-1β when being neutralized with the antibody disclosed herein in an in vivo and/or an in vitro setting, i.e., the antibody completely inhibits the action of IL-1β. “No residual activity” as used herein refers to lower than 2% of the potency assay signal corresponding to the IL-6 release from human fibroblasts induced by 10 pg/ml of IL-1β, e.g., the assay can be carried out in presence of 60 ng/ml of the antibody described herein when compared to antibodies of non-relevant specificity or vehicle control at the same concentration.

The antibody can be cross-reactive with IL-1β from non-human species, such as, without being limited to, cynomolgus IL-1β, rhesus monkey IL-1β, rat IL-1β, murine IL-1β, canine IL-1β, feline IL-1β, marmoset IL-1β, swine IL-1β and/or guinea pig IL-1β. In some embodiments, the antibody is cross-reactive with cynomolgus IL-1β (e.g., recombinantly produced and available from Sino Biological Inc., cat. no. 90010-CNAE), rhesus monkey IL-10 (e.g., recombinantly produced and available from R&D Systems, cat. no. 1318-RL/CF) and/or rat IL-1β (e.g., recombinantly produced and available from Peprotech, cat. no. 400-01B).

In some embodiments, the antibody disclosed herein is a full length immunoglobulin or an antibody fragment (i.e., a Fab, Fab′, F(ab′)₂, scFv fragment, nanobody, VHH or minimal recognition unit). In some embodiments, the antibody is an antibody fragment with a molecular weight of about 50 kDa, or lower, such as about 54 kDa, 40 kDa, 35 kDa, 27 kDa, 26 kDa, 25 kDa, 24 kDa, or 23 kDa. In some embodiments, the antibody or fragment thereof has the VH and VL domains connected in either orientation by a flexible linker (e.g., VL-linker-VH or VH-linker-VL). In some embodiments, the orientation is VL-linker-VH with the light chain variable region being at the N-terminal end and the heavy chain variable region being at the C-terminal end of the polypeptide. The flexible linker typically comprises 10 to about 25 amino acids, e.g., glycine to confer flexibility and/or serines and/or threonines for improved solubility. For example, in some embodiments, a (GGGGS)₄ linker (SEQ ID NO: 9) or a variant thereof is used. Variations of said motif having three to five repeats may also be used. Further suitable linkers are described, e.g., in ALFTHAN, K. Properties of a single-chain antibody containing different linker peptides. Protein Engineering 1995, vol. 8, no. 7, p. 725-731, which are incorporated by reference in its entirety. In some embodiments, the antibody fragment is a scFv fragment, wherein the VL and VH region are connected with a flexible linker such as SEQ ID NO: 9.

In one embodiment, the antibody or fragment thereof comprises a heavy chain variable region comprising the amino acid sequence of SEQ ID NO: 7 or a variant sequence at least 85% identical thereto, e.g., at least 90, 95, 96, 97, 98, or 99% identical thereto. In some embodiments, the antibody or fragment comprises a heavy chain variable region comprising at least 50 contiguous amino acids of the amino acid sequence of SEQ ID NO: 7 or a variant sequence at least 90% identical thereto, e.g., at least 75, 85, or 100 or more contiguous amino acids. Examples of such variant VH sequences include, without being limited to, SEQ ID NO: 121, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 142, SEQ ID NO: 144, SEQ ID NO: 146, SEQ ID NO: 148, SEQ ID NO: 150, or SEQ ID NO: 152.

In one embodiment, the antibody or fragment thereof comprises a light chain variable region comprising the amino acid sequence of SEQ ID NO: 8 or a variant sequence at least 85% identical thereto, e.g., at least 90, 95, 96, 97, 98, or 99% identical thereto. In some embodiments, the antibody or fragment comprises a light chain variable region comprising at least 50 contiguous amino acids of the amino acid sequence of SEQ ID NO: 8 or a variant sequence at least 90% identical thereto, e.g., at least 75, 85, or 100 or more contiguous amino acids. Examples of such variant VL sequences include, without being limited to, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 136, SEQ ID NO: 137, SEQ ID NO: 139 or SEQ ID NO: 153.

In one embodiment, the antibody or fragment thereof comprises a heavy chain variable region comprising the amino acid sequence of SEQ ID NO: 7 or a variant sequence at least 85% identical thereto, e.g., at least 90, 95, 96, 97, 98, or 99% identical thereto, and a light chain variable region comprising the amino acid sequence of SEQ ID NO: 8 or a variant sequence at least 85% identical thereto, e.g., at least 90, 95, 96, 97, 98, or 99% identical thereto. In some embodiments, the antibody or fragment comprises a heavy chain variable region comprising at least 50 contiguous amino acids of the amino acid sequence of SEQ ID NO: 7 or a variant sequence at least 90% identical thereto, e.g., at least 75, 85, or 100 or more contiguous amino acids, and a light chain variable region comprising at least 50 contiguous amino acids of the amino acid sequence of SEQ ID NO: 8 or a variant sequence at least 90% identical thereto, e.g., at least 75, 85, or 100 or more contiguous amino acids. In some embodiments, the heavy chain variable region and the light chain variable region are connected with a sequence linker such as SEQ ID NO: 9.

In some embodiments, the antibody of the invention comprises SEQ ID NO: 10 or variants thereof. In some embodiments, the antibody has at least 85%, 90%, 95%, 96%, 97%, 98%, or 99% sequence similarity with SEQ ID NO: 10. In some embodiments, the antibody has at least 85%, 90%, 95%, 96%, 97%, 98%, or 99% sequence identity with SEQ ID NO: 10.

In some embodiment, the antibody or a fragment thereof comprises at least one of a heavy chain variable region (VH) CDR (e.g., 1, 2, or 3) sequence as set forth in SEQ ID NOS: 1, 2, 3, respectively, or variants thereof. For example, in some embodiments, the antibody or a fragment thereof comprises a heavy chain variable region (VH) CDR-1 sequence as set forth in SEQ ID NO:1, a heavy chain variable region (VH) CDR-2 sequence as set forth in SEQ ID NO: 2, a heavy chain variable region (VH) CDR-3 sequence as set forth in SEQ ID NO: 3, or a portion thereof from the amino acid sequence of SEQ ID NO: 7 or a sequence at least 75% identical thereto, e.g., at least 80, 85, 90, 95, 96, 97, 98, or 99% identical thereto.

In one embodiment, the antibody or a fragment thereof comprises at least one of a light chain variable region (VL) CDR (e.g., 1, 2, or 3) sequence as set forth in SEQ ID NOS: 5, 6, 7, respectively, or variants thereof. For example, in some embodiments, the antibody or a fragment thereof comprises a light chain variable region (VL) CDR-1 sequence as set forth in SEQ ID NO: 4, a light chain variable region (VL) CDR-2 sequence as set forth in SEQ ID NO: 5, a light chain variable region (VL) CDR-3 sequence as set forth in SEQ ID NO: 6, or a portion thereof from the amino acid sequence of SEQ ID NO: 8 or a variant sequence at least 75% identical thereto, e.g., at least 80, 85, 90, 95, 96, 97, 98, or 99% identical thereto.

In some embodiments, the antibody or a fragment thereof comprises at least one of the heavy chain variable region (VH) CDR (e.g., 1, 2, or 3) sequence as set forth in SEQ ID NOS: 1, 2, 3, respectively, or variants thereof and/or at least one light variable chain (VL) CDR (e.g., 1, 2, or 3) sequence as set forth in SEQ ID NOS: 5, 6, 7, respectively, or variants thereof.

In some embodiments, the antibody or fragment thereof is humanized. In some embodiments, the antibody or fragment thereof comprises at least one light chain variable framework region (e.g., FR-L1, -L2, -L3, or L4). For example, in some embodiments, the antibody or fragment thereof comprises at least one light chain variable framework region FR-L1 of SEQ ID NO: 18, FR-L2 of SEQ ID NO: 19; FR-L3 of SEQ ID NO: 20; and/or FR-L4 of SEQ ID NO: 21; or variants thereof.

In some embodiments, the antibody or fragment thereof comprises at least one heavy chain variable framework region (e.g., FR-H1, -H2, -H3, or H4). For example, in some embodiments, the antibody or fragment thereof comprises at least one heavy chain variable framework region FR-H1 of SEQ ID NOS: 22, 26, or 30; FR-H2 of SEQ ID NOS: 23, 27, or 31; FR-H3 of SEQ ID NOS: 24, 28, or 32; and/or FR-H4 of SEQ ID NOS: 25, 29, or 33; or variants thereof.

In some embodiments, the antibody or fragment thereof comprises light chain variable framework region FR-L1 of SEQ ID NO: 18 and SEQ ID NO: 21 and heavy chain variable framework region FR-H1 of SEQ ID NO: 22, FR-H2 of SEQ ID NOS: 23, 27, and 31, FR-H3 of SEQ ID NO: 24, and FR-H4 of SEQ ID NOS: 25 and 26.

In one embodiment, the antibody comprises the VH as set forth in to SEQ ID NO: 7 and the VL as set forth in SEQ ID NO:8. Framework sequences of both SEQ ID NO: 7 and SEQ ID NO:8 are derived from a human immunoglobulin described in WO 03/097697 A (ESBATech AG). Its VH and VL framework sequences have been modified for humanization and stabilization of rabbit antibodies, see, e.g., WO 2009/155726 A (ESBATech, AN ALCON BIOMEDICAL RESEARCH UNIT LLC); BORRAS, L., et al. Generic approach for the generation of stable humanized single-chain Fv fragments from rabbit monoclonal antibodies. Journal of Biological Chemistry 2010, vol. 285, no. 12, p. 9054-9066. In one embodiment, the VL framework of the antibody disclosed herein comprises SEQ ID NOS: 18-21 or variants thereof. Additionally or alternatively, the VH framework of the antibody comprises SEQ ID NOS: 22-25, SEQ ID NOS: 26-29 or SEQ ID NOS: 30-33 or variants thereof, respectively.

In one embodiment, the antibody comprises the VH as set forth in to SEQ ID NO: 146 and the VL as set forth in SEQ NO: 8 or in SEQ ID NO: 145. In some embodiments, the antibody comprises the VH as set forth in to SEQ ID NO: 146 and the VL as set forth in SEQ ID NO: 136.

In certain embodiments, variants of the antibodies or fragments thereof are prepared to improve one or more characteristics of the antibody. For example, it may be desirable to improve antigen binding, antibody-dependent cell-mediated cytotoxicity (ADCC), complement-dependent cytotoxicity (CDC), to increase stability or solubility, to decrease immunogenicity and/or to alter other biological, biochemical or biophysical properties of the antibody. In some embodiments, the variant does not show any improvement over the parent antibody.

Variants of the antibodies provided herein may be prepared by protein and/or chemical engineering, introducing appropriate modifications into the nucleic acid sequence encoding the antibody, or by protein/peptide synthesis. Any combination(s) of deletions, substitutions, additions and insertions can be made to the framework or to the CDRs, provided that the generated antibody possesses the desired characteristics for which it can be screened using appropriate methods. Of particular interest are substitutions, preferably conservative substitutions as described above. Preferred conservative substitutions include:

1. Substituting alanine (A) by valine (V);
2. Substituting arginine (R) by lysine (K);
3. Substituting asparagine (N) by glutamine (Q);
4. Substituting aspartic acid (D) by glutamic acid (E);
5. Substituting cysteine (C) by serine (S);
6. Substituting glutamic acid (E) by aspartic acid (D);
7. Substituting glycine (G) by alanine (A);
8. Substituting histidine (H) by arginine (R) or lysine (K);
9. Substituting isoleucine (I) by leucine (L);
10. Substituting methionine (M) by leucine (L);
11. Substituting phenylalanine (F) by tyrosine (Y);
12. Substituting proline (P) by alanine (A);
13. Substituting serine (S) by threonine (T);
14. Substituting tryptophan (W) by tyrosine (Y);
15. Substituting phenylalanine (F) by tryptophan (W);
and/or
16. Substituting valine (V) by leucine (L)
and vice versa.

The antibody described herein may comprise one or more, such as two, three, four, five, six, seven, eight, nine, ten, eleven, twelve or more of such conservative substitutions. In some embodiments, the antibody described herein may comprise 12 or less of such conservative substitutions, e.g., 11, 10, 9, 8, 7, 6, 5, 4, 3, 2, or 1 of such conservative substitutions.

Non-conservative substitutions may lead to more substantial changes, e.g., with respect to the charge, dipole moment, size, hydrophilicity, hydrophobicity or conformation of the polypeptide. In one embodiment, the antibody comprises one or more, such as two, three, four, five, six, seven, eight, nine, ten, eleven, twelve or more of such non-conservative substitutions. In some embodiments, the antibody described herein may comprise 12 or less of such non-conservative substitutions, e.g., 11, 10, 9, 8, 7, 6, 5, 4, 3, 2, or 1 of such non-conservative substitutions.

Modifications may be present in the CDRs or in the framework sequences. For example, the CDRs provided herein may comprise one, two, three, four, five or even more modifications. For example, the CDR-L1, CDR-L2 and CDR-L3 sequences taken as a whole are at least 75%, 76%, 77%, 78%, 79%, 80%, 85%, 90%, 91%, 92%, 93%, 94%, 95%, 96%, 97%, 98% or 99% identical to the CDRs provided herein, in particular to (i) SEQ ID NOS: 4, 5 and 6, or to (ii) SEQ ID NOS: 161, 162 and 163. Additionally or alternatively, the CDR-H1, CDR-H2 and CDR-H3 sequences taken as a whole are at least 80%, 81%, 82%, 83%, 84%, 95%, 90%, 91%, 92%, 93%, 94%, 95%, 96%, 97%, 98% or 99% identical to the CDRs provided herein, in particular to (i) SEQ ID NOS: 1, 2 and 3, or to (ii) SEQ ID NOS: 155, 156 and 157.

In one embodiment the CDR-L1, CDR-L2, CDR-L3, CDR-H1, CDR-H2 and CDR-H3 taken as a whole are at least 85%, 90%, 91%, 92%, 93%, 94%, 95%, 96%, 97%, 98% or 99% similar to the CDRs provided herein. Additionally or alternatively, the CDR-L1, CDR-L2, CDR-L3, CDR-H1, CDR-H2 and CDR-H3 taken as a whole are at least 85%, 90%, 91%, 92%, 93%, 94%, 95%, 96%, 97%, 98% or 99% similar to the CDRs provided herein.

Therefore, a variant may, e.g., comprise one, two, three, four or five substitutions in SEQ ID NO:4. In some embodiments, substitutions are at positions marked with X in SEQ ID NO: 14. The variant may, e.g., comprise:

(i) alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), tyrosine (Y) at AHo position 32 of the variable light chain;
(ii) alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), serine (S), threonine (T), valine (V), tryptophan (W), tyrosine (Y) at AHo position 33 of the variable light chain; and/or
(iii) glutamic acid (E), phenylalanine (F), glycine (G), methionine (M), asparagine (N), glutamine (Q), serine (S), tryptophan (W), tyrosine (Y) at AHo position 40 of the variable light chain.

Additionally or alternatively, a variant comprises one, two, three, or four substitutions in SEQ ID NO: 5. In some embodiments, substitutions are at positions marked with X in SEQ ID NO: 15. Such variant may, e.g., comprise:

(i) alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), tryptophan (W), tyrosine (Y) at AHo position 58 of the variable light chain; and/or
(ii) alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), tyrosine (Y) at AHo position 69 of the variable light chain.

Additionally or alternatively, a variant comprises one, two, three, four, five or six substitutions in SEQ ID NO: 6. In some embodiments, substitutions are at positions marked with X in SEQ ID NO: 16. For example, such variant may comprise:

(i) alanine (A), cysteine (C), isoleucine (I), asparagine (N), serine (S), threonine (T), valine (V) at AHo position 109 of the variable light chain;
(ii) alanine (A), glycine (G), proline (P), serine (S) at AHo position 111 of the variable light chain;
(iii) alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), tyrosine (Y) at AHo position 112 of the variable light chain;
(iv) alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), tyrosine (Y) at AHo position 135 of the variable light chain; and/or
(v) alanine (A), cysteine (C), aspartic acid (D), glutamic acid (E), phenylalanine (F), glycine (G), histidine (H), isoleucine (I), leucine (L), methionine (M), asparagine (N), proline (P), glutamine (Q), arginine (R), serine (S), threonine (T), valine (V), tryptophan (W), tyrosine (Y) at AHo position 136 of the variable light chain.

Additionally or alternatively, a variant comprises one, two, three, or four substitutions in SEQ ID NO: 1 or in SEQ ID NO: 155. In some embodiments, substitutions are at positions marked with X in SEQ ID NO: 11. Such variant may, e.g., comprise:

(i) alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), tyrosine (Y) at AHo position 33 of the variable heavy chain; and/or
(ii) alanine (A), cysteine (C), aspartic acid (D), glutamic acid (E), phenylalanine (F), glycine (G), histidine (H), isoleucine (I), lysine (K), leucine (L), methionine (M), asparagine (N), glutamine (Q), arginine (R), serine (S), threonine (T), valine (V), tryptophan (W), tyrosine (Y) at AHo position 39 of the variable heavy chain.

Additionally or alternatively, a variant comprises one, two, three, four, five or six substitutions in SEQ ID NO: 2 or in SEQ ID NO: 156. In some embodiments, substitutions are at positions marked with X in SEQ ID NO: 12. For example, the variant may comprise:

(i) alanine (A), cysteine (C), glycine (G), methionine (M) or tyrosine (Y) at AHo position 59 of the variable heavy chain;
(ii) aspartic acid (D), asparagine (N) or proline (P) at AHo position 60 of the variable heavy chain; and/or
(iii) alanine (A), aspartic acid (D), glutamic acid (E), glycine (G), phenylalanine (F), histidine (H), isoleucine (I), lysine (K), leucine (L), methionine (M), asparagine (N), proline (P), serine (S), threonine (T), tryptophan (W) or tyrosine (Y) at AHo position 69 of the variable heavy chain.

Additionally or alternatively, a variant comprises one, two, three, four, five, six, seven, eight, nine, ten or eleven substitutions in SEQ ID NO: 3 or in SEQ ID NO: 157. In some embodiments, substitutions are at positions marked with X in SEQ ID NO: 13. Such variant may, e.g., comprise:

(i) alanine (A), cysteine (C), aspartic acid (D), glutamic acid (E), phenylalanine (F), glycine (G), histidine (H), isoleucine (I), lysine (K), leucine (L), methionine (M), asparagine (N), glutamine (Q), arginine (R), serine (S), threonine (T), valine (V), tryptophan (W), tyrosine (Y) at AHo position 110 of the variable heavy chain;
(ii) alanine (A), cysteine (C), aspartic acid (D), phenylalanine (F), glycine (G), histidine (H), isoleucine (I), lysine (K), methionine (M), asparagine (N), proline (P), glutamine (Q), arginine (R), serine (S), threonine (T), valine (V), tryptophan (W), tyrosine (Y) at AHo position 111 of the variable heavy chain;
(iii) alanine (A), cysteine (C), phenylalanine (F), histidine (H), isoleucine (I), leucine (L), methionine (M), asparagine (N), glutamine (Q), serine (S), threonine (T), valine (V), tyrosine (Y) at AHo position 112 of the variable heavy chain;
(iv) phenylalanine (F) or isoleucine (I) at AHo position 113 of the variable heavy chain;
(v) alanine (A), cysteine (C), glutamic acid (E), glycine (G), serine (S), threonine (T), valine (V) at AHo position 114 of the variable heavy chain;
(vi) alanine (A), glycine (G), methionine (M) or asparagine (N) at AHo position 115 of the variable heavy chain;
(vii) alanine (A), aspartic acid (D), glutamic acid (E), histidine (H), asparagine (N), serine (S), threonine (T) at AHo position 135 of the variable heavy chain;
(viii) alanine (A), cysteine (C), phenylalanine (F), glycine (G), histidine (H), isoleucine (I), leucine (L), methionine (M), asparagine (N), glutamine (Q), serine (S), threonine (T), valine (V), tryptophan (W), tyrosine (Y) at AHo position 136 of the variable heavy chain;
(ix) alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), tyrosine (Y) at AHo position 137 of the variable heavy chain; and/or
(x) alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), tyrosine (Y) at AHo position 138 of the variable heavy chain.

One type of variant is one where one or more entire CDRs are replaced. Typically, the CDR-H3 and CDR-L3 contribute most significantly to antigen binding. For example, the entire CDR-L1, CDR-L2, CDR-H1 and/or CDR-H2 may be replaced by a different CDR of natural or artificial origin. In some embodiments, one or more CDRs are replaced by an alanine-cassette.

In some embodiments, the variant described herein has at least 85%, 90%, 91%, 92%, 93%, 94%, 95%, 96%, 97%, 98%, 99%, or 100% sequence identity to a sequence selected from the group consisting of SEQ ID NO: 10, SEQ ID NO: 73, and SEQ ID NO 82.

In some embodiments, the variant described herein has at least 85%, 90%, 91%, 92%, 93%, 94%, 95%, 96%, 97%, 98%, 99%, or 100% sequence similarity to SEQ ID NO: 10, SEQ ID NO: 73, and SEQ ID NO: 82.

Additionally or alternatively, the VH of the antibody comprises solubility enhancing point mutations. WO2009/155725 (ESBATech, a Novartis company) describes a motif, which has proven to increase the overall solubility of the antibody. The residues are placed at positions located in the interface of the variable domain and the constant domain of an antibody and stabilize antibody fragments, in particular scFv, lacking the constant domain. In particular, one, and/or all three of the following residues are present:

(i) serine (S) at heavy chain amino acid position 12 (according to AHo numbering);
(ii) serine (S) or threonine (T) at heavy chain amino acid position 103 (according to AHo numbering); and/or
(iii) serine (S) or threonine (T) at heavy chain amino acid position 144 (according to AHo numbering).

In one embodiment, the antibody has a serine at VH position 12; a serine at VH position 103; and a threonine at VH position 144 (all AHo numbering). Thus, in one embodiment, the antibody disclosed herein comprises the VH framework sequences of SEQ ID NOS: 30-33 or variants thereof.

In one embodiment, the variant comprises a VL sequence selected from the group consisting of SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 136, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, and SEQ ID NO: 153.

Additionally or alternatively, the variant comprises a VH sequence selected from the group consisting of SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 138, SEQ ID NO: 140, SEQ ID NO: 142, SEQ ID NO: 144, SEQ ID NO: 146, SEQ ID NO: 148, SEQ ID NO: 150, and SEQ ID NO: 152.

Variants may also be prepared by chain shuffling of light and heavy chains. A single light chain can be combined with a library of heavy chains to yield a library of variants. In one embodiment, said single light chain is selected from the group of VL sequences recited above and/or said library of heavy chains comprises one or more of the VH sequences recited above. Likewise, a single heavy chain can be combined with a library of light chains. In one embodiment, said single heavy chain is selected from the group of VH sequences recited above and/or said library of light chains comprises one or more of the VL sequences recited above.

In one embodiment, the variant comprises the VL of SEQ ID NO: 135 and/or the VH of SEQ ID NO: 7, SEQ ID NO: 142, SEQ ID NO: 146, SEQ ID NO: 150, or SEQ ID NO: 152. In one embodiment, the variant comprises SEQ ID NO: 67, SEQ ID NO: 85, SEQ ID NO: 86, SEQ ID NO: 87, or SEQ ID NO: 88. In one embodiment, the variant comprises the VL of SEQ ID NO:136 and/or the VH of SEQ ID NO: 7, SEQ ID NO: 142, SEQ ID NO: 146, SEQ ID NO: 150, or SEQ ID NO: 152. In one embodiment, the variant comprises SEQ ID NO: 68, SEQ ID NO: 81, SEQ ID NO: 82, SEQ ID NO: 83, or SEQ ID NO: 84. In one embodiment, the variant comprises the VL of SEQ ID NO: 137 and/or the VH of SEQ ID NO: 7, SEQ ID NO: 138, SEQ ID NO: 142, SEQ ID NO: 146, SEQ ID NO: 150, or SEQ ID NO: 152. In one embodiment, the variant comprises SEQ ID NO: 69, SEQ ID NO: 92, SEQ ID NO: 93, SEQ ID NO: 94, or SEQ ID NO: 95. In one embodiment, the variant comprises the VL of SEQ ID NO: 139 and/or the VH of SEQ ID NO: 140, SEQ ID NO: 142, SEQ ID NO: 146, SEQ ID NO: 150, or SEQ ID NO: 152. In one embodiment, the variant comprises SEQ ID NO: 70, SEQ ID NO: 77, SEQ ID NO: 78, SEQ ID NO: 79 or SEQ ID NO: 80. In one embodiment, the variant comprises the VL of SEQ ID NO: 141 and/or the VH of SEQ ID NO: 142. In one embodiment, the variant comprises SEQ ID NO: 71. In one embodiment, the variant comprises the VL of SEQ ID NO: 143 and/or the VH of SEQ ID NO: 144. In one embodiment, the variant comprises SEQ ID NO: 72. In one embodiment, the variant comprises the VL of SEQ ID NO: 145 and/or the VH of SEQ ID NO: 146. In one embodiment, the variant comprises SEQ ID NO: 73. In one embodiment, the variant comprises the VL of SEQ ID NO: 147 and/or the VH of SEQ ID NO: 148. In one embodiment, the variant comprises SEQ ID NO: 74. In one embodiment, the variant comprises the VL of SEQ ID NO: 149 and/or the VH of SEQ ID NO: 150. In one embodiment, the variant comprises SEQ ID NO: 75. In one embodiment, the variant comprises the VL of SEQ ID NO: 151 and/or the VH of SEQ ID NO: 152. In one embodiment, the variant comprises SEQ ID NO: 76. In one embodiment, the variant comprises the VL of SEQ ID NO: 8 and/or the VH of SEQ ID NO: 121 or of SEQ ID NO: 122. In one embodiment, the variant comprises SEQ ID NO: 60. In one embodiment, the variant comprises the VL of SEQ ID NO: 153 and/or the VH of SEQ ID NO: 142, SEQ ID NO: 146, or SEQ ID NO: 152. In one embodiment, the variant comprises SEQ ID NO: 89, SEQ ID NO: 90 or SEQ ID NO: 91. In one embodiment, the variant comprises the VL of SEQ ID NO: 8 and/or the VH of SEQ ID NO: 121, SEQ ID NO: 122, SEQ ID NO: 142, SEQ ID NO: 144, SEQ ID NO: 146, SEQ ID NO: 148, SEQ ID NO: 150, or SEQ ID NO: 152. In one embodiment, the variant comprises the VH of SEQ ID NO: 7 and/or the VL of SEQ ID NO: 135, SEQ ID NO: 136, SEQ ID NO: 137, SEQ ID NO: 139, or SEQ ID NO: 153. In one embodiment, the variant comprises a sequence selected from the group consisting of SEQ ID NOS: 34-95 and SEQ ID NO: 154.

The antibodies of the instant invention are particularly stable. As used herein the term “stability” refers to the biophysical property of the antibody to remain monomeric in solution after prolonged incubation and/or incubation at elevated temperature. Unstable antibodies tend to dimerize or oligomerize and even precipitate, thereby decreasing shelf-life and becoming less suitable for pharmaceutical applications.

The antibodies provided herein and in particular the antibody fragment of the invention remains monomeric at least to 75%, at least to 80%, at least to 85%, or at least 93% after being incubated for 1 month at 37° C. at a concentration of 1 mg/ml in PBS at pH 7.2. Additionally or alternatively, the antibody remains monomeric at least to 90%, e.g., at least to 92%, 94%, 96%, 98%, or 100% after 1 month at room temperature at a concentration of 1 mg/ml in PBS at pH 7.2.

The degree of monomers can, e.g., be determined by SEC-HPLC (Size Exclusion Chromatography-High-Performance Liquid Chromatography). A suitable mobile phase for such testing is, e.g., PBS at pH 7.2. The monomer content can be quantified by peak integration of the UV280 signal measured during the protein chromatography. A suitable system is, e.g., a Dionex Summit HPLC controlled by CHROMELEON® 6.5 software that also allows for subsequent chromatogram analysis and peak quantification.

The antibodies disclosed herein, and in particular the monovalent antibody fragment above, are also stable at higher concentrations. For example, they remain monomeric at least to 50%, 55%, 60%, 65%, 70%, or 75% after being incubated for 2 weeks at room temperature and/or 4° C. at a concentration of about 50 mg/ml in PBS at pH 7.2.

Moreover, the antibodies provided herein, and in particular the monovalent antibody fragment above, are particularly soluble and can therefore be highly concentrated without precipitation due to aggregate formation. For example, the antibodies can be concentrated in PBS at pH 7.2 to a concentration of more than 20 mg/ml without precipitation, e.g., to a concentration of 30 mg/ml, 40 mg/ml, 50 mg/ml, 60 mg/ml, or 70 mg/ml in PBS at pH 7.2.

In one embodiment, the antibody has a melting temperature of about 60° C. as determined by differential scanning fluorimetry (DSF), e.g., 65° C., 70° C., 71° C., 72° C., 73° C., or 74° C. This method is based on the properties of certain dyes being fluorescent only in a hydrophobic environment. For example, protein unfolding can be detected as an increase in fluorescence upon binding of the dye SYPRO® Orange to a heat-denatured protein (NIESEN F. H. et al. The use of differential scanning fluorimetry to detect ligand interactions that promote protein stability. Nature Protocols 2007, vol. 2, p. 2212-2221). The stability of a protein can thus be analysed by thermal denaturation.

The antibody may have a theoretical isoelectric point (pI) in the range of 5 to 10, e.g., 7 to 9, e.g., about 8.3. The theoretical pI can, for example, be calculated by using the ProtParam tool on the ExPASy Server (available at web.expasy.org/protparam/; see also GASTEIGER E. et al. Protein Identification and Analysis Tools on the ExPASy Server. (In) The Proteomics Protocols Handbook. Edited by WALKER J. M. Totowa: Humana Press Inc., 2005. ISBN 9781588295934. p. 571-607).

The antibodies described herein are encoded by a single nucleic acid or by two or more nucleic acids, for example each encoding at least one variable region. Knowing the sequence of the antibody or of its parts, cDNAs encoding the polypeptide sequence can be generated by methods well known in the art, e.g., by gene synthesis. For example, in some embodiments, the antibody can be produced by expression in a suitable host cell and can be recovered using standard techniques in the art. In other embodiments, the antibody is produced in a cell-free system. For more methods, see, U.S. Pat. No. 9,404,930 to Grabulovski et al, which is incorporated herein by reference in its entirety.

As a further aspect, the invention provides a pharmaceutical composition comprising the antibody or fragment thereof formulated with a pharmaceutically acceptable carrier; diluent, or recipient. By “pharmaceutically acceptable” it is meant a material that is not biologically or otherwise undesirable, i.e., the material can be administered to a subject without causing any undesirable biological effects such as toxicity. For example, in some embodiments, the pharmaceutical composition comprises a suitable pH, osmolarity, ionic balance, sterility, endotoxin, and/or pyrogen level that is/are suitable for administration to humans. The release of active agent (i.e., antibody) from any formulation, pharmaceutical composition, or device described herein is optionally tunable to the desired release characteristics. For example, in some embodiments, the pharmaceutical composition comprises pharmaceutical acceptable excipients, diluents, and/or carriers suitable for a particular mode of administration. For example, in some embodiments, the pharmaceutical composition comprises components suitable for parenteral or transdermal administration. For example, in some embodiments, the pharmaceutical composition comprises components suitable for subcutaneous or intradermal administration. In some embodiments, the pharmaceutical composition comprises components suitable for transdermal administration, e.g., by using a patch.

In some embodiments, a pharmaceutically acceptable composition in this invention is a formulation that is “auris-acceptable” and/or “auris-pharmaceutically acceptable.” Such a composition comprises a carrier or diluent, which does not abrogate the biological activity or properties of the antibody in reference to the auris interna (or inner ear), and is relatively or is reduced in toxicity to the auris interna (or inner ear). In some embodiments, the pharmaceutical formulation comprises a pH and/or osmolality and/or osmolarity that ensures homeostasis of the target auris structure and is compatible with the microenvironment of the inner ear (e.g., perilymph). For example, a perilymph-suitable osmolarity/osmolality is a practical/deliverable osmolarity/osmolality that maintains the homeostasis of the target auris strucutre during administration of the pharmaceutical composition described herein.

In some embodiments, pharmaceutical compositions described herein are substantially free of a gelling component. Such pharmaceutical compositions can provide immediate release of the antibody when administered locally (e.g., to one or more structures within the ear), enterally, or parenterally. For an auris-pharmaceutically acceptable formulation a suitable viscosity is required for administration to structures within the ear without significantly dripping and/or draining into the Eustachian tube during and/or post administration. Such dripping and/or draining into the Eustachian tube can result in rapid clearance of the antibody present in the formulation and, thus, should be minimized and/or avoided.

In some embodiments, the pharmaceutical composition comprises a gelling component. For example, in some embodiments, the pharmaceutical composition comprises an antibody of the invention in a gel matrix, example of such compositions include but should not be limited to topical gel formulations applied directly to the skin and/or is part of a transdermal patch, and/or is a gel formulation that can be injected subcutaneously, and/or is an auris-acceptable gel formulation where all of the components of the gel formulation are compatible with the targeted auris structure. The viscosity of the gel formulation can vary and is in part determined based on the mode of administration, e.g., topical, subcutaneous, intradermal, or transdermal. In some embodiments, the gel formulation provides controlled release of the antibody to the desired treatment site such as topical or subcutaneous or intradermal areas of the skin or targeted auris structure within the ear.

In general, controlled release drug formulations impart control over the release of drug with respect to the site of release and the time of release within the targeted body part(s). As discussed herein, controlled release refers to immediate release, delayed release, sustained release, extended release, variable release, pulsatile release and bi-modal release. Many advantages are offered by controlled release. First, controlled release of a pharmaceutical agent allows less frequent dosing and thus minimizes repeated treatment. Second, controlled release treatments result in more efficient drug utilization and less of the active drug substance remains as a residue. Third, controlled release offers the possibility of localized drug delivery by placement of a delivery device or formulation at the site of disease. Still further, controlled release offers the opportunity to administer and release two or more different drugs, each having a unique release profile, or to release the same drug at different rates or for different durations, by means of a single dosage unit.

In some embodiments, the pharmaceutical composition (e.g., gel formulation) has an immediate or rapid release component for delivery of the antibody to the desired target site. In other embodiments, the pharmaceutical composition (e.g., gel formulation) has a sustained release component for delivery of the antibody to the desired target site. In some embodiments, pharmaceutical compositions (e.g., gel formulation for auris, subcutaneous, or topical application) are biodegradable. In some embodiments, auris gel formulations include a mucoadhesive excipient to allow adhesion to the external mucous layer of the round window membrane. In some embodiments, auris gel formulations include a penetration enhancer excipient, a viscosity enhancing agent sufficient to provide a viscosity, and/or a bioadhesive agent. In some embodiments, the auris-acceptable pharmaceutical gels also contain co-solvents, preservatives, co-solvents, ionic strength and osmolality adjustors and other excipients in addition to buffering agents. Suitable auris-acceptable water soluble buffering agents are alkali or alkaline earth metal carbonates, phosphates, bicarbonates, citrates, borates, acetates, succinates and the like, such as sodium phosphate, citrate, borate, acetate, bicarbonate, carbonate and tromethamine (TRIS). These agents are present in amounts sufficient to maintain the pH of the system at 7.4±0.2 and preferably, 7.4. As such, the buffering agent is as much as 5% on a weight basis of the total composition.

In some embodiments, the auris gel formulations comprises any type of auris-acceptable gel, such as but not limited to, an auris-acceptable thermoreversible gel, an auris-acceptable hydrogel, an auris-acceptable xerogel, an auris-acceptable release gel, or combinations thereof.

In some embodiments, the pharmaceutical composition does not comprise a gel, but comprises an auris-acceptable microsphere, microcapsule or microparticle, an auris-acceptable in situ forming spongy material, an auris-acceptable liposome, an auris-acceptable nanocapsule, nanoparticle, or nanosphere, an auris-acceptable foam, or combinations thereof.

In general, administration of pharmaceutical compositions, i.e., auris-acceptable gel formulation, occurs on or near the round window membrane via intratympanic injection. In some embodiments, the pharmaceutical composition is administered on or near the round window or the crista fenestrae cochleae through entry via a post-auricular incision and surgical manipulation into or near the round window or the crista fenestrae cochleae area. Alternatively, the pharmaceutical composition is applied via syringe and needle, wherein the needle is inserted through the tympanic membrane and guided to the area of the round window or crista fenestrae cochleae. The pharmaceutical compositions are then deposited on or near the round window or crista fenestrae cochleae for localized treatment of AIED. In other embodiments, the pharmaceutical compositions are applied via microcathethers implanted into the patient, and in other embodiments the formulations are administered via a pump device onto or near the round window membrane. In still further embodiments, the pharmaceutical compositions are applied at or near the round window membrane via a microinjection device. In some embodiments, the pharmaceutical compositions are applied in the tympanic cavity. In some embodiments, the pharmaceutical compositions are applied on the tympanic membrane. In still other embodiments, the pharmaceutical composition is applied onto or in the auditory canal. In some embodiments, such pharmaceutical composition do not significantly drip and/or drain into the Eustachian tube. Thus, in some embodiments, the treatment of AIED comprises local administration of a controlled release pharmaceutical composition, i.e., a controlled release auris-acceptable gel formulation, containing the antibody of the invention. In some embodiments, the pharmaceutical composition has been formulated for intratympanic injection (e.g., into the middle and/or inner ear), but should not be limited to this particular route of administration. Additional suitable pharmaceutical formulations of the invention also include pharmaceutical formulations for other routes of administration, i.e., oral, subcutaneous, intramuscular, intradermal, interlymphatic, intravenous, intranasal, transdermal, and inhalation.

For example, in some embodiments, the antibody of the invention is formulated into a pharmaceutical composition comprising a carrier, excipients, diluents, or combinations thereof, wherein suitable carriers, excipients and/or diluents include, without being limited to: (i) buffers such as phosphate, citrate, or other, organic acids; (ii) antioxidants such as ascorbic acid and tocopherol; (iii) preservatives such as 3-pentanol, hexamethonium chloride, benzalkonium chloride, benzyl alcohol, alkyl paraben, catechol, or cyclohexanol; (iv) amino acids, such as e.g., histidine, arginine; (v) peptides, preferably up to 10 residues such as polylysine; (vi) proteins, such as bovine or human serum albumin; (vii) hydrophilic polymers such as polyvinylpyrrolidone; (viii) monosaccharides, disaccharides, polysaccharides and/or other carbohydrates including glucose, mannose, sucrose, mannitol, trehalose, sorbitol, aminodextran or polyamidoamines; (ix) chelating agents, e.g., EDTA; (x) salt-forming ions such as sodium; (xi) metal complexes (e.g., Zn-protein complexes); and/or (xii) ionic and non-ionic surfactants such as TWEEN™, PLURONICS™ or polyethylene glycol (PEG). For example, the carrier will typically be a liquid, such as sterile pyrogen-free water, pyrogen-free phosphate-buffered saline solution, bacteriostatic water, or Cremophor EL[R] (BASF, Parsippany, N.J.). Such compositions are suitable, for example, for parental administration including but not limited to subcutaneous, intraderaml, and/or transdermal administration. For other methods of administration, the carrier can be either solid or liquid.

In some embodiments, the pharmaceutical formulation of the invention can optionally comprise medicinal agents, therapeutic agents, carriers, adjuvants, dispersing agents, diluents, and the like. The compounds of the invention can be formulated for administration in a pharmaceutical carrier in accordance with known techniques. See, e.g., Remington, The Science And Practice of Pharmacy (22^{nd} Ed.), 2015. In the manufacture of a pharmaceutical formulation according to the invention, the antibody or fragment thereof (including the physiologically acceptable salts thereof) is typically admixed with, inter alia, an acceptable carrier. The carrier can be a solid or a liquid, or both, and is preferably formulated with the antibody or fragment thereof as a unit-dose formulation. For example, the compound can be formulated as an injectable containing from 0.01 or 0.5% to 95% or 99% by weight of the antibody or fragment thereof. One or more agents and/or compounds can be incorporated in the pharmaceutical formulation of the invention, which can be prepared by any of the well-known techniques of pharmacy.

A further aspect of the invention relates to kits for use in the methods of the invention. The kit can comprise the antibody or fragment thereof of the invention in a form suitable for administration to a subject or in a form suitable for compounding into a formulation. The kit can further comprise other components, such as therapeutic agents, carriers, buffers, containers, devices for administration, and the like. The kit can further comprise labels and/or instructions, e.g., for treatment of a disorder. Such labeling and/or instructions can include, for example, information concerning the amount, frequency and method of administration of the antibodies or fragments thereof of the invention.

Having described the present invention, the same will be explained in greater detail in the following examples, which are included herein for illustration purposes only, and which are not intended to be limiting to the invention.

### Example 1

### Characterization of DLX-2323

DLX-2323 (SEQ ID NO:10) is a humanized single chain antibody fragment (scFv) that binds to human Interleukin-1β (IL-1β) with high affinity. DLX-2323 is expressed in E. coli and accumulates as inclusion bodies after induction of protein production inside the cytoplasm. The protein consists of 249 amino acid residues; the calculated molecular weight of the native protein (two disulfide bonds will be formed) is 25,663.6 Da.

DLX-2323 has an IC₅₀ of 3 pM for inhibition of IL-1β-induced IL-6 release and is 10-20 times more potent than canakinumab, a whole anti-IL-1β antibody approved for treatment of cryopyrin-associated periodic syndrome (CAPS). It is 10 times more potent than the natural IL-1 receptor antagonist (IL-1RA). DLX-2323 is highly selective for IL-1β with no activity against IL-1α, IL-18, IL-33, IL-36, TNFα, or IL-6. DLX-2323 is specific for primate IL-1β (human, cynomolgus and rhesus monkey). It does not inhibit canine, porcine, guinea pig, rat or mouse IL-1β.

Comparison of Recombinant Human Interleukin 1 Receptor Antagonist (rhlL-Lra), ILARIS (Canakinumab), and DLX-2323 in a IL-1β Neutralization Assay

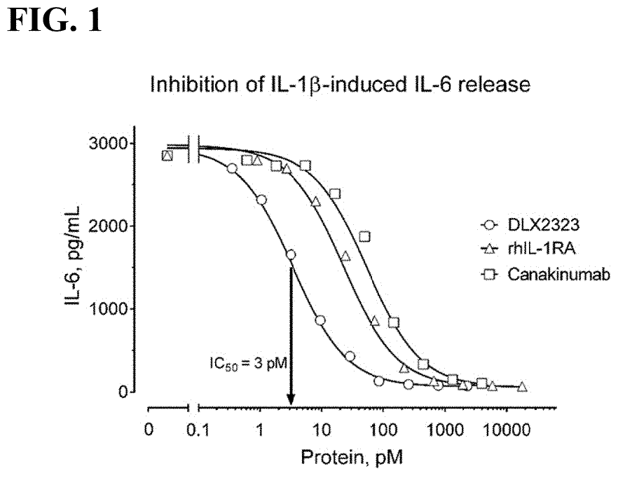

Normal human dermal human fibroblasts in fibroblast basal medium were used in the assay. Varying concentrations of (rhIL-1ra), canakinumab, and DLX-2323 were added to the wells prior to addition of 10 pg/mL recombinant human IL-1β. Release of IL-6 in response to IL-1β exposure was assessed by ELISA. Decreased concentrations of IL-6 reflect neutralization of rhIL-1β. In this assay, DLX-2323 was more potent (leftward shift) than rhIL-1ra and canakinumab (FIG. 1) with an IC₅₀ of 3 pM.

Affinity of scFv DLX-2323

DLX-2323 was measured against an Antigen (IL-1β) to determine the dissociation constant, Kd, using the Sapidyne Instruments Kinetic Exclusion Assay. Kd is a specific type of equilibrium constant that measures the propensity of a larger object to separate (dissociate) reversibly into smaller components. A Kd analysis requires immobilization of one interaction partner (the titrated binding partner) to a solid phase which is then used as a probe to capture the other interaction partner. For each experiment, one of the binding partners is titrated in a background of the captured binding partner (CBP) and allowed to reach equilibrium. The solutions are then briefly exposed to the solid phase and a portion of free CBP is captured. The captured CBP is then labelled with a fluorescent secondary molecule. The signals generated from CBP are used to calculate the Kd value which is directly proportional to the concentration of free CBP in the equilibrated sample. The results of the experiments are presented in Table 1.

TABLE 1
DLX-2323 to IL-1β Affinity

| | |
|---|---|
| Kd | 756 fM |
| 95% Confidence Interval | 285 fM-1.46 pM |
| Concentration Calculated for: | DLX-2323 |
| Activity | 68.2% |
| 95% Confidence Interval | 61.2%-76.2% |
| kon (M⁻¹s⁻¹) | 7.55 × 10⁶ |
| 95% Confidence Interval (M⁻¹s⁻¹) | 6.78 × 10⁶ to 8.41 × 10⁶ |
| Koff(s⁻¹) | 5.70 × 10⁻⁶ |

Cross-Reactivity Analysis for DLX-2323 (Cynomolgus and Rhesus Monkey IL-1β, hIL1a, hTNFα)

The binding of DLX-2323 to rhIL-1β, rhesus macaque IL-1β, cynomolgus IL-1β, human IL-1ra, and human TNFα was assessed in vitro. Plate wells were coated with each protein and incubated overnight at 4° C.

DLX-2323 was added to the wells at varying concentrations, as were positive control antibodies. DLX-2323 specifically bound to human IL-1β and recognized both cynomolgus and rhesus IL-1β in a direct ELISA. It did not bind human IL-1ra or human TNFα (Table 2).

Cross-Reactivity Analysis for DLX-2323 (IL-1β of Different Species hIL-1ra, hIL-6)

The binding of DLX-2323 to rhIL-1β, recombinant canine IL-1β, recombinant porcine IL-1β, recombinant guinea pig IL-1β, recombinant mouse IL-1β, recombinant rat IL-1β, recombinant human IL-1ra, and recombinant human IL-6 was assessed in vitro. Recombinant human IL-1β 1 mg/ml was diluted with the coating buffer to a final concentration of 2 μg/ml. 50 μl was added to the designated wells. Canine IL-1β, porcine/swine IL-1β, guinea pig IL-1β, human IL-1ra and human IL-6 were diluted in the coating buffer and added to distinct wells on the plates. Plates were incubated overnight at 4° C. DLX-2323 was added to the wells at varying concentrations, as were positive control antibodies. The bound scFvs and control antibodies were detected with Protein L-Peroxidase L-HRP (1:3000), rabbit anti-goat HRP (1:5000) and streptavidin HRP (1:3000).

DLX-2323 specifically bound to human IL-1β in a concentration dependent manner. DLX-2323 did not show any detectable binding to canine IL-1β, porcine IL-1β, guinea pig IL-1β, mouse IL-1β, human IL-1ra and human IL-6. Weak binding (10-20%) was observed for rat IL-1β. (Table 2).

TABLE 2
Species and IL-1 Family Cross-reactivity

| | DLX2323 |
|---|---|
| Target proteins | IC₅₀ 2-3 pM |
| Human recombinant IL-1β | + |
| Human natural IL-1β | + |
| Canine rIL-1β | 0 |
| Cynomolgus rIL-1β | + |
| Rhesus monkey rIL-1β | + |
| Porcine rIL-1β | 0 |
| Guinea pig rIL-1β | 0 |
| Rat rIL-1β | 10-20% |
| Mouse rIL-1β | 0 |
| Human rlL-1ra | 0 |
| Human rIL-1α | 0 |
| Mouse rIL-1α | 0 |
| Human rIL-18 | 0 |
| Human rIL-33 | 0 |
| Human rTNFα | 0 |
| Human rIL-6 | 0 |
| Human rIL-36ra | 0 |
| Mouse rIL-6 | 0 |

### DLX-2323 Penetration Through Reconstructed Human Epidermis (RHE)

DLX-2323 administered via IT injection must penetrate the round window membrane of the cochlea to distribute into the perilymph and reach its projected site of action. A study of penetration through reconstructed human epidermis was conducted and demonstrated permeability through a barrier similar to round window membrane.

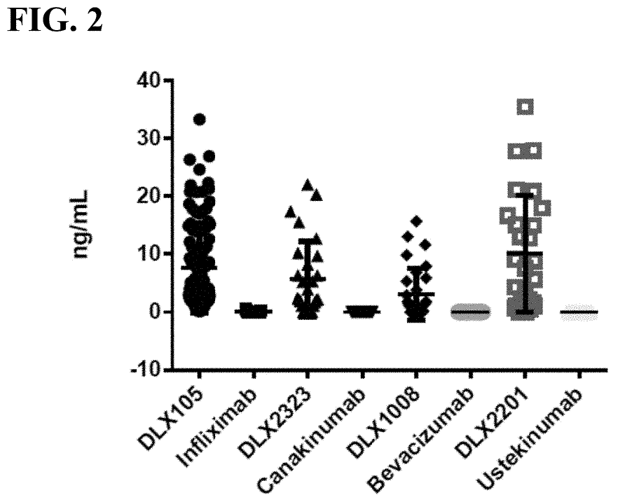

An RHE in vitro system was used to study the skin permeation behaviour of DLX-2323. Different scFvs (DLX-105, DLX-2323, DLX-1008 and DLX-2201) and corresponding monoclonal IgG antibodies (Infliximab, Canakinumab, Bevacizumab and Ustekinumab) were applied in buffered saline solution (FIG. 2) onto reconstructed human epidermis tissues. DLX-2323 permeated this epidermal barrier unlike the full IL-1β antibody canakinumab. This demonstrated that DLX-2323 can penetrate an epithelial barrier similar to round window membrane.

### Efficacy Study—Inhibition of IL-1β-Induced Inflammation by Test Antibodies in Balb/c Mice

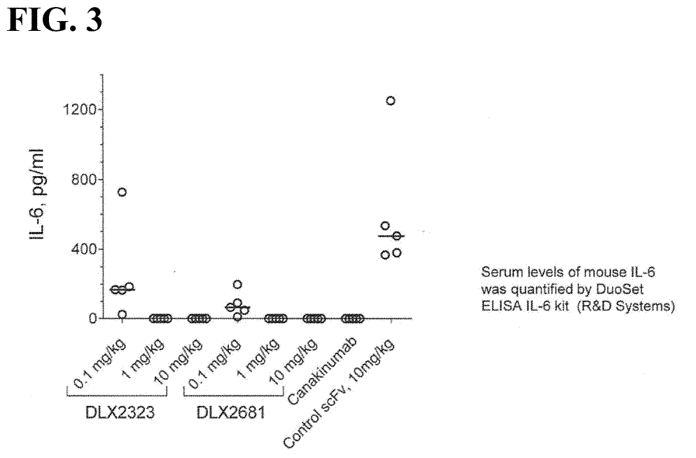

DLX-2323 has no affinity for mouse IL-1β, but injection of human IL-1β results in release of mouse IL-6 into the blood. This allows testing for neutralization of human IL-1β in vivo. Five groups of healthy mice were tested, including 3 DLX-2323 dose groups and 2 control groups. Five animals per group were treated with a single dose of DLX-2323, DLX-2681 (another proprietary scFv IL-1β antibody) or a DLX antibody without affinity for human IL-1β (negative control) and canakinumab (positive control, a whole human monoclonal antibody targeted at IL-1β). Administration of test compounds was performed IV (Canakinumab), IP (DLX antibodies) or SC (rhlL-1β) prior to injection of human IL-1β. Both DLX-2323 and canakinumab completely blocked the release of mouse IL-6. DLX-2323 was effective in a dose-dependent manner from 0.1 to 10 mg/kg (FIG. 3). The negative control antibody was not effective.

It will be understood by those of skill in the art that numerous and various modifications can be made without departing from the spirit of the present invention. Therefore, it should be clearly understood that the forms of the present invention are illustrative only and are not intended to limit the scope of the present invention.

All publications, patent applications, patents, patent publications, sequences identified by GenBank® database accession numbers and other references cited herein are incorporated by reference in their entireties for the teachings relevant to the sentence and/or paragraph in which the reference is presented.

The foregoing is illustrative of the present invention, and is not to be construed as limiting thereof. The invention is defined by the following claims, with equivalents of the claims to be included therein.

Sequence listing

SEQ ID NO: 1 - VH CDR1
FSLSSAAMA
SEQ ID NO: 2 - VH CDR2
IIYDSASTYYASWAKG
SEQ ID NO: 3 - VH CDR3
ERAIFSGDFVL
SEQ ID NO: 4 - VL CDR1
QASQSIDNWLS
SEQ ID NO: 5 - VL CDR2
RASTLAS
SEQ ID NO: 6 - VL CDR3
QNTGGGVSIA
SEQ ID NO: 7 - VH
EVQLVESGGGLVQPGGSLRLSCTASGFSLSSAAMAWVRQAPGKGLEWVGI
IYDSASTYYASWAKGRFTISRDTSKNTVYLQMNSLRAEDTAVYYCARERA
IFSGDFVLWGQGTLVTVSS
SEQ ID NO: 8 - VL
EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLG
SEQ ID NO: 9 - linker
GGGGSGGGGSGGGGSGGGGS
SEQ ID NO: 10 - DLX2323
EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSGDFVLWGQGTLVTVSS
SEQ ID NO: 11 - CDR variant of VH CDR1
FSLSXXAMA
SEQ ID NO: 12 - CDR variant of VH CDR2
IIXXSASTXYASWAKG
SEQ ID NO: 13 - CDR variant of VH CDR3
EXXXXXXXXXX
SEQ ID NO: 14 - CDR variant of VL CDR1
QASQSIXXXLS
SEQ ID NO: 15 - CDR variant of VL CDR2
XASXLAS
SEQ ID NO: 16 - CDR variant of VL CDR3
QNXGXXXXIA
SEQ ID NO: 17 - DNA sequence of DLX2323
GAAATTGTTATGACCCAGAGCCCGAGCACCCTGAGCGCAAGCGTTGGTGA
TCGTGTGATTATTACCTGTCAGGCAAGCCAGAGCATTGATAATTGGCTGA
GCTGGTATCAGCAGAAACCGGGTAAAGCACCGAAACTGCTGATTTATCGT
GCAAGCACCCTGGCAAGCGGTGTTCCGAGCCGTTTTAGCGGTAGCGGTAG
TGGTGCAGAATTTACCCTGACCATTAGCAGCCTGCAGCCGGATGATTTTG
CAACCTATTATTGTCAGAATACCGGTGGTGGTGTTAGCATTGCATTTGGT
CAGGGCACCAAACTGACCGTTCTGGGTGGTGGCGGTGGATCCGGTGGGGG
TGGTAGCGGAGGTGGTGGTTCAGGCGGTGGTGGCAGCGAAGTTCAGCTGG
TTGAAAGTGGTGGTGGTCTGGTTCAGCCTGGTGGTAGCCTGCGTCTGAGC
TGTACCGCAAGCGGTTTTAGCCTGAGCAGCGCAGCAATGGCATGGGTTCG
TCAGGCACCTGGTAAAGGTCTGGAATGGGTTGGTATTATCTATGATAGCG
CAAGCACCTATTATGCAAGCTGGGCAAAAGGTCGTTTTACCATTAGCCGT
GATACCAGTAAAAATACCGTTTACCTGCAGATGAATAGTCTGCGTGCAGA
GGATACCGCAGTGTATTATTGTGCACGTGAACGTGCAATTTTCAGCGGTG
ATTTTGTTCTGTGGGGTCAGGGAACCCTGGTTACCGTTAGCAGC
SEQ ID NO: 18 - FR-L1 of FW1.4
EIVMTQSPSTLSASVGDRVIITC
SEQ ID NO: 19 - FR-L2 of FW1.4
WYQQKPGKAPKLLIY
SEQ ID NO: 20 - FR-L3 of FW1.4
GVPSRFSGSGSGAEFTLTISSLQPDDFATYYC
SEQ ID NO: 21 - FR-L4 of FW1.4
FGQGTKLTVLG
SEQ ID NO: 22 - FR-H1 of rFW1.4
EVQLVESGGGLVQPGGSLRLSCTASG
SEQ ID NO: 23 - FR-H2 of rFW1.4
WVRQAPGKGLEWVG
SEQ ID NO: 24 - FR-H3 of rFW1.4
RFTISRDTSKNTVYLQMNSLRAEDTAVYYCAR
SEQ ID NO: 25 - FR-H4 of rFW1.4
WGQGTLVTVSS
SEQ ID NO: 26 - FR-H1 of rFW1.4(V2)
EVQLVESGGGLVQPGGSLRLSCTVSG
SEQ ID NO: 27 - FR-H2 of rFW1.4(V2)
WVRQAPGKGLEWVG
SEQ ID NO: 28 - FR-H3 of rFW1.4(V2)
RFTISKDTSKNTVYLQMNSLRAEDTAVYYCAR
SEQ ID NO: 29 - FR-H4 of rFW1.4(V2)
WGQGTLVTVSS
SEQ ID NO: 30 - FR-H1 of rFW1.4-SST
EVQLVESGGGSVQPGGSLRLSCTASG
SEQ ID NO: 31 - FR-H2 of rFW1.4-SST
WVRQAPGKGLEWVG
SEQ ID NO: 32 - FR-H3 of rFW1.4-SST
RFTISRDTSKNTVYLQMNSLRAEDTASYYCAR
SEQ ID NO: 33 - FR-H4 of rFW1.4-SST
WGQGTTVTVSS
SEQ ID NO: 34 - CDR-L1_D32X
EIVMTQSPSTLSASVGDRVIITCQASQSIXNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSGDFVLWGQGTLVTVSS

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W) and tyrosine (Y).

CDR-L1_N33X

SEQ ID NO: 35

EIVMTQSPSTLSASVGDRVIITCQASQSIDXWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSGDFVLWGQGTLVTVSS

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), serine (S), threonine (T), valine (V), tryptophan (W) and tyrosine (Y).

CDR-L1_W40X

SEQ ID NO: 36

EIVMTQSPSTLSASVGDRVIITCQASQSIDNXLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSGDFVLWGQGTLVTVSS

Preferably, X is selected from the group consisting of glutamic acid (E), phenylalanine (F), glycine (G), methionine (M), asparagine (N), glutamine (Q), serine (S), tryptophan (W) and tyrosine (Y).

CDR-L2_R58X

SEQ ID NO: 37

EIVMTQSPSTLSASVGDRVIITCQASQSIDNWLSWYQQKPGKAPKLLIYX
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSGDFVLWGQGTLVTVSS

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), tryptophan (W) and tyrosine (Y).

CDR-L2_T69X

SEQ ID NO: 38

EIVMTQSPSTLSASVGDRVIITCQASQSIDNWLSWYQQKPGKAPKLLIYR
ASXLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSGDFVLWGQGTLVTVSS

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W) and tyrosine (Y).

CDR-L3_T109X

SEQ ID NO: 39

EIVMTQSPSTLSASVGDRVIITCQASQSIDNWLSWYQQKPGKAPKLLIYR
ASTLASGVPSRFSGSGSGAEFTLTISSLQPDDFATYYCQNXGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSGDFVLWGQGTLVTVSS

Preferably, X is selected from the group consisting of alanine (A), cysteine (C), isoleucine (I), asparagine (N), serine (S), threonine (T) and valine (V).

CDR-L3_G111X

SEQ ID NO: 40

EIVMTQSPSTLSASVGDRVIITCQASQSIDNWLSWYQQKPGKAPKLLIYR
ASTLASGVPSRFSGSGSGAEFTLTISSLQPDDFATYYCQNTGXGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSGDFVLWGQGTLVTVSS

Preferably, X is selected from the group consisting of alanine (A), glycine (G), proline (P) and serine (S).

CDR-L3_G112X

SEQ ID NO: 41

EIVMTQSPSTLSASVGDRVIITCQASQSIDNWLSWYQQKPGKAPKLLIYR
ASTLASGVPSRFSGSGSGAEFTLTISSLQPDDFATYYCQNTGGXVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSGDFVLWGQGTLVTVSS

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W) and tyrosine (Y).

CDR-L3_V135X

SEQ ID NO: 42

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGXSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSGDFVLWGQGTLVTVSS

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W) and tyrosine (Y).

CDR-L3_S136X

SEQ ID NO: 43

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVXIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSGDFVLWGQGTLVTVSS

Preferably, X is selected from the group consisting of alanine (A), cysteine (C), aspartic acid (D), glutamic acid (E), phenylalanine (F), glycine (G), histidine (H), isoleucine (I), leucine (L), methionine (M), asparagine (N), proline (P), glutamine (Q), arginine (R), serine (S), threonine (T), valine (V), tryptophan (W) and tyrosine (Y).

CDR-H1_S33X

SEQ ID NO: 44

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSXAAMAWVRQAPGKGLEWVGIIYDSASTYYASWAKGRFTISR
DTSKNTVYLQMNSLRAEDTAVYYCARERAIFSGDFVLWGQGTLVTVSS

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W) and tyrosine (Y).

CDR-H1_A39X

SEQ ID NO: 45

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSXAMAWVRQAPGKGLEWVGIIYDSASTYYASWAKGRFTISR
DTSKNTVYLQMNSLRAEDTAVYYCARERAIFSGDFVLWGOGTLVTVSS

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), glutamine (Q), arginine (R), serine (S), threonine (T), valine (V), tryptophan (W) and tyrosine (Y).

CDR-H2_Y59X

SEQ ID NO: 46

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIXDSASTYYASWAKGRFTISR
DTSKNTVYLQMNSLRAEDTAVYYCARERAIFSGDFVLWGQGTLVTVSS

Preferably, X is selected from the group consisting of alanine (A), cysteine (C), glycine (G), methionine (M) and tyrosine (Y).

CDR-H2_D60X

SEQ ID NO: 47

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYXSASTYYASWAKGRFTISR
DTSKNTVYLQMNSLRAEDTAVYYCARERAIFSGDFVLWGQGTLVTVSS

Preferably, X is selected from the group consisting of aspartic acid (D), asparagine (N) and proline (P).

CDR-H2_Y69X

SEQ ID NO: 48

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTXYASWAKGRFTISR
DTSKNTVYLQMNSLRAEDTAVYYCARERAIFSGDFVLWGQGTLVTVSS

Preferably, X is selected from the group consisting of alanine (A), aspartic acid (D), glutamic acid (E), glycine (G), phenylalanine (F), histidine (H), isoleucine (I), lysine (K), leucine (L), methionine (M), proline (P), asparagine (N), serine (S), threonine (T), tryptophan (W) and tyrosine (Y).

CDR-H3_R110X

SEQ ID NO: 49

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCAREXAIFSGDFVLWGQGTLVTVSS

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), glutamine (Q), arginine (R), serine (S), threonine (T), valine (V), tryptophan (W) and tyrosine (Y).

CDR-H3_A111X

SEQ ID NO: 50

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERXIFSGDFVLWGQGTLVTVSS

Preferably, X is selected from the group consisting of alanine (A), cysteine (C), aspartic acid (D), phenylalanine (F), glycine (G), histidine (H), isoleucine (I), lysine (K), methionine (M), asparagine (N), proline (P), glutamine (Q), arginine (R), serine (S), threonine (T), valine (V), tryptophan (W) and tyrosine (Y).

CDR-H3_I112X

SEQ ID NO: 51

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAXFSGDFVLWGQGTLVTVSS

Preferably, X is selected from the group consisting of alanine (A), cysteine (C), phenylalanine (F), histidine (H), isoleucine (I), leucine (L), methionine (M), asparagine (N), glutamine (Q), serine (S), threonine (T), valine (V) and tyrosine (Y).

CDR-H3_F113X

SEQ ID NO: 52

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIXSGDFVLWGQGTLVTVSS

Preferably, X is selected from the group consisting of phenylalanine (F) and isoleucine (I).

CDR-H3_S114X

SEQ ID NO: 53

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFXGDFVLWGQGTLVTVSS

Preferably, X is selected from the group consisting of alanine (A), cysteine (C), glutamic acid (E), glycine (G), serine (S), threonine (T) and valine (V).

CDR-H3_G115X

SEQ ID NO: 54

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSXDFVLWGQGTLVTVSS

Preferably, X is selected from the group consisting of alanine (A), glycine (G), methionine (M) and asparagine (N).

CDR-H3_D135X

SEQ ID NO: 55

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSGXFVLWGQGTLVTVSS

Preferably, X is selected from the group consisting of alanine (A), aspartic acid (D), glutamic acid (E), histidine (H), asparagine (N), serine (S) and threonine (T).

CDR-H3_F136X

SEQ ID NO: 56

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSGDXVLWGQGTLVTVSS

Preferably, X is selected from the group consisting of alanine (A), cysteine (C), phenylalanine (F), glycine (G), histidine (H), isoleucine (I), leucine (L), methionine (M), asparagine (N), glutamine (Q), serine (S), threonine (T), valine (V), tryptophan (W) and tyrosine (Y).

CDR-H3_V137X

SEQ ID NO: 57

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSGDFXLWGQGTLVTVSS

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W) and tyrosine (Y).

CDR-H3_L138X

SEQ ID NO: 58

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSGDFVXWGQGTLVTVSS

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W) and tyrosine (Y).

DLX2464

SEQ ID NO: 59

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERQIFSGDMAGWGQGTLVTVSS
DLX2465

SEQ ID NO: 60

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERNIFSGDMDLWGQGTLVTVSS
DLX2466

SEQ ID NO: 61

EIVMTQSPSTLSASVGDRVIITCQASQSIGKYLSWYQQKPGKAPKLLIYR
ASTLASGVPSRFSGSGSGAEFTLTISSLQPDDFATYYCQNAGGGVSIAFG
QGTKLTVLGGGGGSGGGGSGGGGSGGGGSEVQLVESGGGLVQPGGSLRLS
CTASGFSLSSDAMAWVRQAPGKGLEWVGIIYDSASTYYASWAKGRFTISR
DTSKNTVYLQMNSLRAEDTAVYYCARERNIFSGDMAGWGQGTLVTVSS
DLX2467

SEQ ID NO: 62

EIVMTQSPSTLSASVGDRVIITCQASQSIHNWLSWYQQKPGKAPKLLIYR
ASNLASGVPSRFSGSGSGAEFTLTISSLQPDDFATYYCQNTGGGSSIAFG
QGTKLTVLGGGGGSGGGGSGGGGSGGGGSEVQLVESGGGLVQPGGSLRLS
CTASGFSLSRAAMAWVRQAPGKGLEWVGIIYDSASTYYASWAKGRFTISR
DTSKNTVYLQMNSLRAEDTAVYYCARERMIFSGDFVLWGQGTLVTVSS
DLX2468

SEQ ID NO: 63

EIVMTQSPSTLSASVGDRVIITCQASQSIGNYLSWYQQKPGKAPKLLIYR
ASTLASGVPSRFSGSGSGAEFTLTISSLQPDDFATYYCQNAGGGTSIAFG
QGTKLTVLGGGGGSGGGGSGGGGSGGGGSEVQLVESGGGLVQPGGSLRLS
CTASGFSLSSSAMAWVRQAPGKGLEWVGIIYDSASTYYASWAKGRFTISR
DTSKNTVYLQMNSLRAEDTAVYYCARERNIFSGDMVLWGQGTLVTVSS
DLX2475

SEQ ID NO: 64

EIVMTQSPSTLSASVGDRVIITCQASQSIDKWLSWYQQKPGKAPKLLIYQ
ASTLASGVPSRFSGSGSGAEFTLTISSLQPDDFATYYCQNTGGGVHIAFG
QGTKLTVLGGGGGSGGGGSGGGGSGGGGSEVQLVESGGGLVQPGGSLRLS
CTASGFSLSSYAMAWVRQAPGKGLEWVGIIYDSASTYYASWAKGRFTISR
DTSKNTVYLQMNSLRAEDTAVYYCARERAIFSGDFKLWGQGTLVTVSS
DLX2476

SEQ ID NO: 65

EIVMTQSPSTLSASVGDRVIITCQASQSISSWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERDIFSGDFVGWGQGTLVTVSS
DLX2480

SEQ ID NO: 66

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGINIAFG
QGTKLTVLGGGGGSGGGGSGGGGSGGGGSEVQLVESGGGLVQPGGSLRLS
CTASGFSLSDAAMAWVRQAPGKGLEWVGIIYDSASTYYASWAKGRFTISR
DTSKNTVYLQMNSLRAEDTAVYYCARERQIFSGDFVLWGQGTLVTVSS
DLX2543

SEQ ID NO: 67

EIVMTQSPSTLSASVGDRVTITCQASQSISSWLSWYQQKPGKAPKLLIYK
ASTLASGVPSRFSGSGSGTEFTLTISSLQPDDFATYYCQNAGGGVSIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSGDFVLWGQGTLVTVSS
DLX2529

SEQ ID NO: 68

EIVMTQSPSTLSASVGDRVIITCRASQSIGNWLSWYQQKPGKAPKLLIYR
ASNLASGVPSRFSGSGSGAEFTLTISSLQPEDFATYYCQNTGGGINIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSGDFVLWGQGTLVTVSS
DLX2547

SEQ ID NO: 69

ADIVMTQSPSTLSASVGDRVTITCQASQSISSYLSWYQQKPGKAPKLLIY
RASTLASGVPSRFSGSGSGAEFTLTISSLQPDDFATYYCQNTGGGINIAF
GQGTKLEIKRGGGGSGGGGSGGGGSGGGGSEVQLVESGGGLVQPGGSLRL
SCTASGFSLSSAAMAWVRQAPGKGLEWVGIIYDSASTYYASWAKGRFTIS
RDTSKNTVYLQMNSLRAEDTAVYYCARERAIFSGDFVLWGQGTLVTVSS
DLX2528

SEQ ID NO: 70

EIVMTQSPSTLSASVGDRVTITCQASQSIGNWLAWYQQKPGKAPKLLIYQ
ASNLASGVPSRFSGSGSGTDFTLTISSLQPDDFATYYCQNAGGATTIAFG
QGTKLTVLGGGGGSGGGGSGGGGSGGGGSEVQLVESGGGLVQPGGSLRLS
CTASGFSLSSAAMAWVRQAPGKGLEWVGIIYDSASTYYASWAKGRFTISR
DTSKNTVYLQMNSLRAEDTAVYYCARERAIFSGDFVLWGQGTLVTVSS
DLX2585

SEQ ID NO: 71

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTVSGFSLSSYAMSWVRQAPGKGLEWVGIIYDSASTYYASWAKGRFTISK
DTSKNTVYLQMNSLRAEDTAVYYCARERAIFSGDFDYWGQGTLVTVSS
DLX2545

SEQ ID NO: 72

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTASGFSLSSAAMAWVRQAPGKGLEWIGIIYDSASTYYASWAKGRFTISR
DTSKNTLYLQMNSLRAEDTAVYFCARERNIFSGDMVLWGQGTTVTVSS
DLX2531

SEQ ID NO: 73

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGNVQPGGSLRLS
CTASGFSLSNSAMAWVRQAPGKGLEWVGIIYDSASTYYASWAKGRFTISR
DNSKNTVYLQMNSLRAEDTATYYCARERAIFSGDFALWGQGTLVTVSS
DLX2586

SEQ ID NO: 74

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTVSGFSLSSYAMSWVRQAPGKGLEWIGIIYDSASTYYASWAKGRFTISK
DTSKNTVYLQMNSLRAEDTAVYFCARERQIFSGDMDGWGQGTLVTVSS
DLX2530

SEQ ID NO: 75

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGNVQPGGSLRLS
CTASGFSLSDAAMAWVRQAPGKGLEWVGIIYDSASTFYASWAKGRFTISR
DNSKNTLYLQMNSLRAEDTATYYCARERNIFSGDMALWGQGTTVTVSS
DLX2548

SEQ ID NO: 76

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLGGGGGSGGGGSGGGGSGGGGSEVQLVESGGGLVQPGGSLRLS
CTVSGFSLSSYAMSWVRQAPGKGLEWIGIIYDSASTYYASWAKGRFTISK
DTSKNTLYLQMNSLRAEDTAVYFCARERQIFSGDMDGWGQGTTVTVSS
DLX2676

SEQ ID NO: 77

EIVMTQSPSTLSASVGDRVTITCQASQSIGNWLAWYQQKPGKAPKLLIYQ
ASNLASGVPSRFSGSGSGTDFTLTISSLQPDDFATYYCQNAGGATTIAFG
QGTKLTVLGGGGGSGGGGSGGGGSGGGGSEVQLVESGGGNVQPGGSLRLS
CTASGFSLSDAAMAWVRQAPGKGLEWVGIIYDSASTFYASWAKGRFTISR
DNSKNTLYLQMNSLRAEDTATYYCARERNIFSGDMALWGQGTTVTVSS
DLX2677

SEQ ID NO: 78

EIVMTQSPSTLSASVGDRVTITCQASQSIGNWLAWYQQKPGKAPKLLIYQ
ASNLASGVPSRFSGSGSGTDFTLTISSLQPDDFATYYCQNAGGATTIAFG
QGTKLTVLGGGGGSGGGGSGGGGSGGGGSEVQLVESGGGNVQPGGSLRLS
CTASGFSLSNSAMAWVRQAPGKGLEWVGIIYDSASTYYASWAKGRFTISR
DNSKNTVYLQMNSLRAEDTATYYCARERAIFSGDFALWGQGTLVTVSS
DLX2678

SEQ ID NO: 79

EIVMTQSPSTLSASVGDRVTITCQASQSIGNWLAWYQQKPGKAPKLLIYQ
ASNLASGVPSRFSGSGSGTDFTLTISSLQPDDFATYYCQNAGGATTIAFG
QGTKLTVLGGGGGSGGGGSGGGGSGGGGSEVQLVESGGGLVQPGGSLRLS
CTVSGFSLSSYAMSWVRQAPGKGLEWIGIIYDSASTYYASWAKGRFTISK
DTSKNTLYLQMNSLRAEDTAVYFCARERQIFSGDMDGWGQGTTVTVSS
DLX2679

SEQ ID NO: 80

EIVMTQSPSTLSASVGDRVTITCQASQSIGNWLAWYQQKPGKAPKLLIYQ
ASNLASGVPSRFSGSGSGTDFTLTISSLQPDDFATYYCQNAGGATTIAFG
QGTKLTVLGGGGGSGGGGSGGGGSGGGGSEVQLVESGGGLVQPGGSLRLS
CTVSGFSLSSYAMSWVRQAPGKGLEWVGIIYDSASTYYASWAKGRFTISK
DTSKNTVYLQMNSLRAEDTAVYYCARERAIFSGDFDYWGQGTLVTVSS
DLX2680

SEQ ID NO: 81

EIVMTQSPSTLSASVGDRVIITCRASQSIGNWLSWYQQKPGKAPKLLIYR
ASNLASGVPSRFSGSGSGAEFTLTISSLQPEDFATYYCQNTGGGINIAFG
QGTKLTVLGGGGGSGGGGSGGGGSGGGGSEVQLVESGGGNVQPGGSLRLS
CTASGFSLSDAAMAWVRQAPGKGLEWVGIIYDSASTFYASWAKGRFTISR
DNSKNTLYLQMNSLRAEDTATYYCARERNIFSGDMALWGQGTTVTVSS
DLX2681

SEQ ID NO: 82

EIVMTQSPSTLSASVGDRVIITCRASQSIGNWLSWYQQKPGKAPKLLIYR
ASNLASGVPSRFSGSGSGAEFTLTISSLQPEDFATYYCQNTGGGINIAFG
QGTKLTVLGGGGGSGGGGSGGGGSGGGGSEVQLVESGGGNVQPGGSLRLS
CTASGFSLSNSAMAWVRQAPGKGLEWVGIIYDSASTYYASWAKGRFTISR
DNSKNTVYLQMNSLRAEDTATYYCARERAIFSGDFALWGQGTLVTVSS
DLX2682

SEQ ID NO: 83

EIVMTQSPSTLSASVGDRVIITCRASQSIGNWLSWYQQKPGKAPKLLIYR
ASNLASGVPSRFSGSGSGAEFTLTISSLQPEDFATYYCQNTGGGINIAFG
QGTKLTVLGGGGGSGGGGSGGGGSGGGGSEVQLVESGGGLVQPGGSLRLS
CTVSGFSLSSYAMSWVRQAPGKGLEWIGIIYDSASTYYASWAKGRFTISK
DTSKNTLYLQMNSLRAEDTAVYFCARERQIFSGDMDGWGQGTTVTVSS
DLX2683

SEQ ID NO: 84

EIVMTQSPSTLSASVGDRVIITCRASQSIGNWLSWYQQKPGKAPKLLIYR
ASNLASGVPSRFSGSGSGAEFTLTISSLQPEDFATYYCQNTGGGINIAFG
QGTKLTVLGGGGGSGGGGSGGGGSGGGGSEVQLVESGGGLVQPGGSLRLS
CTVSGFSLSSYAMSWVRQAPGKGLEWVGIIYDSASTYYASWAKGRFTISK
DTSKNTVYLQMNSLRAEDTAVYYCARERAIFSGDFDYWGQGTLVTVSS
DLX2684

SEQ ID NO: 85

EIVMTQSPSTLSASVGDRVTITCQASQSISSWLSWYQQKPGKAPKLLIYK
ASTLASGVPSRFSGSGSGTEFTLTISSLQPDDFATYYCQNAGGGVSIAFG
QGTKLTVLGGGGGSGGGGSGGGGSGGGGSEVQLVESGGGNVQPGGSLRLS
CTASGFSLSDAAMAWVRQAPGKGLEWVGIIYDSASTFYASWAKGRFTISR
DNSKNTLYLQMNSLRAEDTATYYCARERNIFSGDMALWGQGTTVTVSS
DLX2685

SEQ ID NO: 86

EIVMTQSPSTLSASVGDRVTITCQASQSISSWLSWYQQKPGKAPKLLIYK
ASTLASGVPSRFSGSGSGTEFTLTISSLQPDDFATYYCQNAGGGVSIAFG
QGTKLTVLGGGGGSGGGGSGGGGSGGGGSEVQLVESGGGNVQPGGSLRLS
CTASGFSLSNSAMAWVRQAPGKGLEWVGIIYDSASTYYASWAKGRFTISR
DNSKNTVYLQMNSLRAEDTATYYCARERAIFSGDFALWGQGTLVTVSS
DLX2686

SEQ ID NO: 87

EIVMTQSPSTLSASVGDRVTITCQASQSISSWLSWYQQKPGKAPKLLIYK
ASTLASGVPSRFSGSGSGTEFTLTISSLQPDDFATYYCQNAGGGVSIAFG
QGTKLTVLGGGGGSGGGGSGGGGSGGGGSEVQLVESGGGLVQPGGSLRLS
CTVSGFSLSSYAMSWVRQAPGKGLEWIGIIYDSASTYYASWAKGRFTISK
DTSKNTLYLQMNSLRAEDTAVYFCARERQIFSGDMDGWGQGTTVTVSS
DLX2687

SEQ ID NO: 88

EIVMTQSPSTLSASVGDRVTITCQASQSISSWLSWYQQKPGKAPKLLIYK
ASTLASGVPSRFSGSGSGTEFTLTISSLQPDDFATYYCQNAGGGVSIAFG
QGTKLTVLGGGGGSGGGGSGGGGSGGGGSEVQLVESGGGLVQPGGSLRLS
CTVSGFSLSSYAMSWVRQAPGKGLEWVGIIYDSASTYYASWAKGRFTISK
DTSKNTVYLQMNSLRAEDTAVYYCARERAIFSGDFDYWGQGTLVTVSS
DLX2689

SEQ ID NO: 89

ADIVMTQSPSTLSASVGDRVTITCQASQSISSYLSWYQQKPGKAPKLLIY
KASTLASGVPSRFSGSGSGTDFTLTISSLQPEDFATYYCQNAGGGINIAF
GQGTKVEIKRGGGGSGGGGSGGGGSGGGGSEVQLVESGGGNVQPGGSLRL
SCTASGFSLSNSAMAWVRQAPGKGLEWVGIIYDSASTYYASWAKGRFTIS
RDNSKNTVYLQMNSLRAEDTATYYCARERAIFSGDFALWGQGTLVTVSS
DLX2690

SEQ ID NO: 90

ADIVMTQSPSTLSASVGDRVTITCQASQSISSYLSWYQQKPGKAPKLLIY
KASTLASGVPSRFSGSGSGTDFTLTISSLQPEDFATYYCQNAGGGINIAF
GQGTKVEIKRGGGGSGGGGSGGGGSGGGGSEVQLVESGGGLVQPGGSLRL
SCTVSGFSLSSYAMSWVRQAPGKGLEWIGIIYDSASTYYASWAKGRFTIS
KDTSKNTLYLQMNSLRAEDTAVYFCARERQIFSGDMDGWGQGTTVTVSS
DLX2691

SEQ ID NO: 91

ADIVMTQSPSTLSASVGDRVTITCQASQSISSYLSWYQQKPGKAPKLLIY
KASTLASGVPSRFSGSGSGTDFTLTISSLQPEDFATYYCQNAGGGINIAF
GQGTKVEIKRGGGGSGGGGSGGGGSGGGGSEVQLVESGGGLVQPGGSLRL
SCTVSGFSLSSYAMSWVRQAPGKGLEWVGIIYDSASTYYASWAKGRFTIS
KDTSKNTVYLQMNSLRAEDTAVYYCARERAIFSGDFDYWGQGTLVTVSS
DLX2692

SEQ ID NO: 92

ADIVMTQSPSTLSASVGDRVTITCQASQSISSYLSWYQQKPGKAPKLLIY
RASTLASGVPSRFSGSGSGAEFTLTISSLQPDDFATYYCQNTGGGINIAF
GQGTKLEIKRGGGGSGGGGSGGGGSGGGGSEVQLVESGGGNVQPGGSLRL
SCTASGFSLSDAAMAWVRQAPGKGLEWVGIIYDSASTFYASWAKGRFTIS
RDNSKNTLYLQMNSLRAEDTATYYCARERNIFSGDMALWGQGTTVTVSS
DLX2693

SEQ ID NO: 93

ADIVMTQSPSTLSASVGDRVTITCQASQSISSYLSWYQQKPGKAPKLLIY
RASTLASGVPSRFSGSGSGAEFTLTISSLQPDDFATYYCQNTGGGINIAF
GQGTKLEIKRGGGGSGGGGSGGGGSGGGGSEVQLVESGGGNVQPGGSLRL
SCTASGFSLSNSAMAWVRQAPGKGLEWVGIIYDSASTYYASWAKGRFTIS
RDNSKNTVYLQMNSLRAEDTATYYCARERAIFSGDFALWGQGTLVTVSS
DLX2694

SEQ ID NO: 94

ADIVMTQSPSTLSASVGDRVTITCQASQSISSYLSWYQQKPGKAPKLLIY
RASTLASGVPSRFSGSGSGAEFTLTISSLQPDDFATYYCQNTGGGINIAF
GQGTKLEIKRGGGGSGGGGSGGGGSGGGGSEVQLVESGGGLVQPGGSLRL
SCTVSGFSLSSYAMSWVRQAPGKGLEWIGIIYDSASTYYASWAKGRFTIS
KDTSKNTLYLQMNSLRAEDTAVYFCARERQIFSGDMDGWGQGTTVTVSS
DLX2695

SEQ ID NO: 95

ADIVMTQSPSTLSASVGDRVTITCQASQSISSYLSWYQQKPGKAPKLLIY
RASTLASGVPSRFSGSGSGAEFTLTISSLQPDDFATYYCQNTGGGINIAF
GQGTKLEIKRGGGGSGGGGSGGGGSGGGGSEVQLVESGGGLVQPGGSLRL
SCTVSGFSLSSYAMSWVRQAPGKGLEWVGIIYDSASTYYASWAKGRFTIS
KDTSKNTVYLQMNSLRAEDTAVYYCARERAIFSGDFDYWGQGTLVTVSS
VL CDR-L1_D32X

SEQ ID NO: 96

EIVMTQSPSTLSASVGDRVIITCQASQSIXNWLSWYQQKPGKAPKLLIYR
ASTLASGVPSRFSGSGSGAEFTLTISSLQPDDFATYYCQNTGGGVSIAFG
QGTKLTVLG

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W) and tyrosine (Y).

VL CDR-L1_N33X

SEQ ID NO: 97

EIVMTQSPSTLSASVGDRVIITCQASQSIDXWLSWYQQKPGKAPKLLIYR
ASTLASGVPSRFSGSGSGAEFTLTISSLQPDDFATYYCQNTGGGVSIAFG
QGTKLTVLG

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), serine (S), threonine (T), valine (V), tryptophan (W) and tyrosine (Y).

VL CDR-L1_W40X

SEQ ID NO: 98

EIVMTQSPSTLSASVGDRVIITCQASQSIDNXLSWYQQKPGKAPKLLIYR
ASTLASGVPSRFSGSGSGAEFTLTISSLQPDDFATYYCQNTGGGVSIAFG
QGTKLTVLG

Preferably, X is selected from the group consisting of glutamic acid (E), phenylalanine (F), glycine (G), methionine (M), asparagine (N), glutamine (Q), serine (S), tryptophan (W) and tyrosine (Y).

VL CDR-L2_R58X

SEQ ID NO: 99

EIVMTQSPSTLSASVGDRVIITCQASQSIDNWLSWYQQKPGKAPKLLIYX
ASTLASGVPSRFSGSGSGAEFTLTISSLQPDDFATYYCQNTGGGVSIAFG
QGTKLTVLG

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), tryptophan (W) and tyrosine (Y).

VL CDR-L2_T69X

SEQ ID NO: 100

EIVMTQSPSTLSASVGDRVIITCQASQSIDNWLSWYQQKPGKAPKLLIYR
ASXLASGVPSRFSGSGSGAEFTLTISSLQPDDFATYYCQNTGGGVSIAFG
QGTKLTVLG

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W) and tyrosine (Y).

VL CDR-L3_T109X

SEQ ID NO: 101

EIVMTQSPSTLSASVGDRVIITCQASQSIDNWLSWYQQKPGKAPKLLIYR
ASTLASGVPSRFSGSGSGAEFTLTISSLQPDDFATYYCQNXGGGVSIAFG
QGTKLTVLG

Preferably, X is selected from the group consisting of alanine (A), cysteine (C), isoleucine (I), asparagine (N), serine (S), threonine (T) and valine (V).

VL CDR-L3_G111X

SEQ ID NO: 102

EIVMTQSPSTLSASVGDRVIITCQASQSIDNWLSWYQQKPGKAPKLLIYR
ASTLASGVPSRFSGSGSGAEFTLTISSLQPDDFATYYCQNTGXGVSIAFG
QGTKLTVLG

Preferably, X is selected from the group consisting of alanine (A), glycine (G), proline (P) and serine (S).

VL CDR-L3_G112X

SEQ ID NO: 103

EIVMTQSPSTLSASVGDRVIITCQASQSIDNWLSWYQQKPGKAPKLLIYR
ASTLASGVPSRFSGSGSGAEFTLTISSLQPDDFATYYCQNTGGXVSIAFG
QGTKLTVLG

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W) and tyrosine (Y).

VL CDR-L3_V135X

SEQ ID NO: 104

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGXSIAFG
QGTKLTVLG

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W) and tyrosine (Y).

VL CDR-L3_S136X

SEQ ID NO: 105

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVXIAFG
QGTKLTVLG

Preferably, X is selected from the group consisting of alanine (A), cysteine (C), aspartic acid (D), glutamic acid (E), phenylalanine (F), glycine (G), histidine (H), isoleucine (I), leucine (L), methionine (M), asparagine (N), proline (P), glutamine (Q), arginine (R), serine (S), threonine (T), valine (V), tryptophan (W) and tyrosine (Y).

VH CDR-H1_S33X

SEQ ID NO: 106

EVQLVESGGGLVQPGGSLRLSCTASGFSLSXAAMAWVRQAPGKGLEWVGI
IYDSASTYYASWAKGRFTISRDTSKNTVYLQMNSLRAEDTAVYYCARERA
IFSGDFVLWGQGTLVTVSS

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W) and tyrosine (Y).

VH CDR-H1_A39X

SEQ ID NO: 107

EVQLVESGGGLVQPGGSLRLSCTASGFSLSSXAMAWVRQAPGKGLEWVGI
IYDSASTYYASWAKGRFTISRDTSKNTVYLQMNSLRAEDTAVYYCARERA
IFSGDFVLWGQGTLVTVSS

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), glutamine (Q), arginine (R), serine (S), threonine (T), valine (V), tryptophan (W) and tyrosine (Y).

VH CDR-H2_Y59X

SEQ ID NO: 108

EVQLVESGGGLVQPGGSLRLSCTASGFSLSSAAMAWVRQAPGKGLEWVGI
IXDSASTYYASWAKGRFTISRDTSKNTVYLQMNSLRAEDTAVYYCARERA
IFSGDFVLWGQGTLVTVSS

Preferably, X is selected from the group consisting of alanine (A), cysteine (C), glycine (G), methionine (M) and tyrosine (Y).

VH CDR-H2_D60X

SEQ ID NO: 109

EVQLVESGGGLVQPGGSLRLSCTASGFSLSSAAMAWVRQAPGKGLEWVGI
IYXSASTYYASWAKGRFTISRDTSKNTVYLQMNSLRAEDTAVYYCARERA
IFSGDFVLWGQGTLVTVSS

Preferably, X is selected from the group consisting of aspartic acid (D), asparagine (N) and proline (P).

VH CDR-H2_Y69X

SEQ ID NO: 110

EVQLVESGGGLVQPGGSLRLSCTASGFSLSSAAMAWVRQAPGKGLEWVGI
IYDSASTXYASWAKGRFTISRDTSKNTVYLQMNSLRAEDTAVYYCARERA
IFSGDFVLWGQGTLVTVSS

Preferably, X is selected from the group consisting of alanine (A), aspartic acid (D), glutamic acid (E), glycine (G), phenylalanine (F), histidine (H), isoleucine (I), lysine (K), leucine (L), methionine (M), proline (P), asparagine (N), serine (S), threonine (T), tryptophan (W) and tyrosine (Y).

VH CDR-H3_R110X

SEQ ID NO: 111

EVQLVESGGGLVQPGGSLRLSCTASGFSLSSAAMAWVRQAPGKGLEWVGI
IYDSASTYYASWAKGRFTISRDTSKNTVYLQMNSLRAEDTAVYYCAREXA
IFSGDFVLWGQGTLVTVSS

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), glutamine (Q), arginine (R), serine (S), threonine (T), valine (V), tryptophan (W) and tyrosine (Y).

VH CDR-H3_A111X

SEQ ID NO: 112

EVQLVESGGGLVQPGGSLRLSCTASGFSLSSAAMAWVRQAPGKGLEWVGI
IYDSASTYYASWAKGRFTISRDTSKNTVYLQMNSLRAEDTAVYYCARERX
IFSGDFVLWGQGTLVTVSS

Preferably, X is selected from the group consisting of alanine (A), cysteine (C), aspartic acid (D), phenylalanine (F), glycine (G), histidine (H), isoleucine (I), lysine (K), methionine (M), asparagine (N), proline (P), glutamine (Q), arginine (R), serine (S), threonine (T), valine (V), tryptophan (W) and tyrosine (Y).

VH CDR-H3_I112X

SEQ ID NO: 113

EVQLVESGGGLVQPGGSLRLSCTASGFSLSSAAMAWVRQAPGKGLEWVGI
IYDSASTYYASWAKGRFTISRDTSKNTVYLQMNSLRAEDTAVYYCARERA
XFSGDFVLWGQGTLVTVSS

Preferably, X is selected from the group consisting of alanine (A), cysteine (C), phenylalanine (F), histidine (H), isoleucine (I), leucine (L), methionine (M), asparagine (N), glutamine (Q), serine (S), threonine (T), valine (V) and tyrosine (Y).

VH CDR-H3_F113X

SEQ ID NO: 114

EVQLVESGGGLVQPGGSLRLSCTASGFSLSSAAMAWVRQAPGKGLEWVGI
IYDSASTYYASWAKGRFTISRDTSKNTVYLQMNSLRAEDTAVYYCARERA
IXSGDFVLWGQGTLVTVSS

Preferably, X is selected from the group consisting of phenylalanine (F) and isoleucine (I).

VH CDR-H3_S114X

SEQ ID NO: 115

EVQLVESGGGLVQPGGSLRLSCTASGFSLSSAAMAWVRQAPGKGLEWVGI
IYDSASTYYASWAKGRFTISRDTSKNTVYLQMNSLRAEDTAVYYCARERA
IFXGDFVLWGQGTLVTVSS

Preferably, X is selected from the group consisting of alanine (A), cysteine (C), glutamic acid (E), glycine (G), serine (S), threonine (T) and valine (V).

VH CDR-H3_G115X

SEQ ID NO: 116

EVQLVESGGGLVQPGGSLRLSCTASGFSLSSAAMAWVRQAPGKGLEWVGI
IYDSASTYYASWAKGRFTISRDTSKNTVYLQMNSLRAEDTAVYYCARERA
IFSXDFVLWGQGTLVTVSS

Preferably, X is selected from the group consisting of alanine (A), glycine (G), methionine (M) and asparagine (N).

VH CDR-H3_D135X

SEQ ID NO: 117

EVQLVESGGGLVQPGGSLRLSCTASGFSLSSAAMAWVRQAPGKGLEWVGI
IYDSASTYYASWAKGRFTISRDTSKNTVYLQMNSLRAEDTAVYYCARERA
IFSGXFVLWGQGTLVTVSS

Preferably, X is selected from the group consisting of alanine (A), aspartic acid (D), glutamic acid (E), histidine (H), asparagine (N), serine (S) and threonine (T).

VH CDR-H3_F136X

SEQ ID NO: 118

EVQLVESGGGLVQPGGSLRLSCTASGFSLSSAAMAWVRQAPGKGLEWVGI
IYDSASTYYASWAKGRFTISRDTSKNTVYLQMNSLRAEDTAVYYCARERA
IFSGDXVLWGQGTLVTVSS

Preferably, X is selected from the group consisting of alanine (A), cysteine (C), phenylalanine (F), glycine (G), histidine (H), isoleucine (I), leucine (L), methionine (M), asparagine (N), glutamine (Q), serine (S), threonine (T), valine (V), tryptophan (W) and tyrosine (Y).

VH CDR-H3_V137X

SEQ ID NO: 119

EVQLVESGGGLVQPGGSLRLSCTASGFSLSSAAMAWVRQAPGKGLEWVGI
IYDSASTYYASWAKGRFTISRDTSKNTVYLQMNSLRAEDTAVYYCARERA
IFSGDFXLWGQGTLVTVSS

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W) and tyrosine (Y).

VH CDR-H3_L138X

SEQ ID NO: 120

EVQLVESGGGLVQPGGSLRLSCTASGFSLSSAAMAWVRQAPGKGLEWVGI
IYDSASTYYASWAKGRFTISRDTSKNTVYLQMNSLRAEDTAVYYCARERA
IFSGDFVXWGQGTLVTVSS

Preferably, X is selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W) and tyrosine (Y).

VH DLX2464

SEQ ID NO: 121

EVQLVESGGGLVQPGGSLRLSCTASGFSLSSAAMAWVRQAPGKGLEWVGI
IYDSASTYYASWAKGRFTISRDTSKNTVYLQMNSLRAEDTAVYYCARERQ
IFSGDMAGWGQGTLVTVSS
VH DLX2465

SEQ ID NO: 122

EVQLVESGGGLVQPGGSLRLSCTASGFSLSSAAMAWVRQAPGKGLEWVGI
IYDSASTYYASWAKGRFTISRDTSKNTVYLQMNSLRAEDTAVYYCARERN
IFSGDMDLWGQGTLVTVSS
VL DLX2466

SEQ ID NO: 123

EIVMTQSPSTLSASVGDRVIITCQASQSIGKYLSWYQQKPGKAPKLLIYR
ASTLASGVPSRFSGSGSGAEFTLTISSLQPDDFATYYCQNAGGGVSIAFG
QGTKLTVLG
VH DLX2466

SEQ ID NO: 124

EVQLVESGGGLVQPGGSLRLSCTASGFSLSSDAMAWVRQAPGKGLEWVGI
IYDSASTYYASWAKGRFTISRDTSKNTVYLQMNSLRAEDTAVYYCARERN
IFSGDMAGWGQGTLVTVSS
VL DLX2467

SEQ ID NO: 125

EIVMTQSPSTLSASVGDRVIITCQASQSIHNWLSWYQQKPGKAPKLLIYR
ASNLASGVPSRFSGSGSGAEFTLTISSLQPDDFATYYCQNTGGGSSIAFG
QGTKLTVLG
VH DLX2467

SEQ ID NO: 126

EVQLVESGGGLVQPGGSLRLSCTASGFSLSRAAMAWVRQAPGKGLEWVGI
IYDSASTYYASWAKGRFTISRDTSKNTVYLQMNSLRAEDTAVYYCARERM
IFSGDFVLWGQGTLVTVSS
VL DLX2468

SEQ ID NO: 127

EIVMTQSPSTLSASVGDRVIITCQASQSIGNYLSWYQQKPGKAPKLLIYR
ASTLASGVPSRFSGSGSGAEFTLTISSLQPDDFATYYCQNAGGGTSIAFG
QGTKLTVLG
VH DLX2468

SEQ ID NO: 128

EVQLVESGGGLVQPGGSLRLSCTASGFSLSSSAMAWVRQAPGKGLEWVGI
IYDSASTYYASWAKGRFTISRDTSKNTVYLQMNSLRAEDTAVYYCARERN
IFSGDMVLWGQGTLVTVSS
VL DLX2475

SEQ ID NO: 129

EIVMTQSPSTLSASVGDRVIITCQASQSIDKWLSWYQQKPGKAPKLLIYQ
ASTLASGVPSRFSGSGSGAEFTLTISSLQPDDFATYYCQNTGGGVHIAFG
QGTKLTVLG
VH DLX2475

SEQ ID NO: 130

EVQLVESGGGLVQPGGSLRLSCTASGFSLSSYAMAWVRQAPGKGLEWVGI
IYDSASTYYASWAKGRFTISRDTSKNTVYLQMNSLRAEDTAVYYCARERA
IFSGDFKLWGQGTLVTVSS
VL DLX2476

SEQ ID NO: 131

EIVMTQSPSTLSASVGDRVIITCQASQSISSWLSWYQQKPGKAPKLLIYR
ASTLASGVPSRFSGSGSGAEFTLTISSLQPDDFATYYCQNTGGGVSIAFG
QGTKLTVLG
VH DLX2476

SEQ ID NO: 132

EVQLVESGGGLVQPGGSLRLSCTASGFSLSSAAMAWVRQAPGKGLEWVGI
IYDSASTYYASWAKGRFTISRDTSKNTVYLQMNSLRAEDTAVYYCARERD
IFSGDFVGWGQGTLVTVSS
VL DLX2480

SEQ ID NO: 133

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGINIAFG
QGTKLTVLG
VH DLX2480

SEQ ID NO: 134

EVQLVESGGGLVQPGGSLRLSCTASGFSLSDAAMAWVRQAPGKGLEWVGI
IYDSASTYYASWAKGRFTISRDTSKNTVYLQMNSLRAEDTAVYYCARERQ
IFSGDFVLWGQGTLVTVSS
VL DLX2543

SEQ ID NO: 135

EIVMTQSPSTLSASVGDRVTITCQASQSISSWLSWYQQKPGKAPKLLIYK
ASTLASGVPSRFSGSGSGTEFTLTISSLQPDDFATYYCQNAGGGVSIAFG
QGTKLTVLG
VL DLX2529

SEQ ID NO: 136

EIVMTQSPSTLSASVGDRVIITCRASQSIGNWLSWYQQKPGKAPKLLIYR
ASNLASGVPSRFSGSGSGAEFTLTISSLQPEDFATYYCQNTGGGINIAFG
QGTKLTVLG
VL DLX2547

SEQ ID NO: 137

ADIVMTQSPSTLSASVGDRVTITCQASQSISSYLSWYQQKPGKAPKLLIY
RASTLASGVPSRFSGSGSGAEFTLTISSLQPDDFATYYCQNTGGGINIAF
GQGTKLEIKR
VH DLX2547

SEQ ID NO: 138

EVQLVESGGGLVQPGGSLRLSCTASGFSLSSAAMAWVRQAPGKGLEWVGI
IYDSASTYYASWAKGRFTISRDTSKNTVYLQMNSLRAEDTAVYYCARERA
IFSGDFVLWGQGTLVTVSS
VL DLX2528

SEQ ID NO: 139

EIVMTQSPSTLSASVGDRVTITCQASQSIGNWLAWYQQKPGKAPKLLIYQ
ASNLASGVPSRFSGSGSGTDFTLTISSLQPDDFATYYCQNAGGATTIAFG
QGTKLTVLG
VH DLX2528

SEQ ID NO: 140

EVQLVESGGGLVQPGGSLRLSCTASGFSLSSAAMAWVRQAPGKGLEWVGI
IYDSASTYYASWAKGRFTISRDTSKNTVYLQMNSLRAEDTAVYYCARERA
IFSGDFVLWGQGTLVTVSS
VL DLX2585

SEQ ID NO: 141

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLG
VH DLX2585

SEQ ID NO: 142

EVQLVESGGGLVQPGGSLRLSCTVSGFSLSSYAMSWVRQAPGKGLEWVGI
IYDSASTYYASWAKGRFTISKDTSKNTVYLQMNSLRAEDTAVYYCARERA
IFSGDFDYWGQGTLVTVSS
VL DLX2545

SEQ ID NO: 143

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLG
VH DLX2545

SEQ ID NO: 144

EVQLVESGGGLVQPGGSLRLSCTASGFSLSSAAMAWVRQAPGKGLEWIGI
IYDSASTYYASWAKGRFTISRDTSKNTLYLQMNSLRAEDTAVYFCARERN
IFSGDMVLWGQGTTVTVSS
VL DLX2531

SEQ ID NO: 145

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLG
VH DLX2531

SEQ ID NO: 146

EVQLVESGGGNVQPGGSLRLSCTASGFSLSNSAMAWVRQAPGKGLEWVGI
IYDSASTYYASWAKGRFTISRDNSKNTVYLQMNSLRAEDTATYYCARERA
IFSGDFALWGQGTLVTVSS
VL DLX2586

SEQ ID NO: 147

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLG
VH DLX2586

SEQ ID NO: 148

EVQLVESGGGLVQPGGSLRLSCTVSGFSLSSYAMSWVRQAPGKGLEWIGI
IYDSASTYYASWAKGRFTISKDTSKNTVYLQMNSLRAEDTAVYFCARERQ
IFSGDMDGWGQGTLVTVSS
VL DLX2530

SEQ ID NO: 149

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLG
VH DLX2530

SEQ ID NO: 150

EVQLVESGGGNVQPGGSLRLSCTASGFSLSDAAMAWVRQAPGKGLEWVGI
IYDSASTFYASWAKGRFTISRDNSKNTLYLQMNSLRAEDTATYYCARERN
IFSGDMALWGQGTTVTVSS
VL DLX2548

SEQ ID NO: 151

EIVMTQSPSTLSASVGDRVIITCQASQSIDNWLSWYQQKPGKAPKLLIYR
ASTLASGVPSRFSGSGSGAEFTLTISSLQPDDFATYYCQNTGGGVSIAFG
QGTKLTVLG
VH DLX2548

SEQ ID NO: 152

EVQLVESGGGLVQPGGSLRLSCTVSGFSLSSYAMSWVRQAPGKGLEWIGI
IYDSASTYYASWAKGRFTISKDTSKNTLYLQMNSLRAEDTAVYFCARERQ
IFSGDMDGWGQGTTVTVSS
VL DLX2544

SEQ ID NO: 153

ADIVMTQSPSTLSASVGDRVTITCQASQSISSYLSWYQQKPGKAPKLLIY
KASTLASGVPSRFSGSGSGTDFTLTISSLQPEDFATYYCQNAGGGINIAF
GQGTKVEIKR
DLX2544

SEQ ID NO: 154

ADIVMTQSPSTLSASVGDRVTITCQASQSISSYLSWYQQKPGKAPKLLIY
KASTLASGVPSRFSGSGSGTDFTLTISSLQPEDFATYYCQNAGGGINIAF
GQGTKVEIKRGGGGSGGGGSGGGGSGGGGSEVQLVESGGGLVQPGGSLRL
SCTASGFSLSSAAMAWVRQAPGKGLEWVGIIYDSASTYYASWAKGRFTIS
RDTSKNTVYLQMNSLRAEDTAVYYCARERAIFSGDFVLWGQGTLVTVSS
CDR-H1 of DLX2531

SEQ ID NO: 155

FSLSNSAMA
CDR-H2 of DLX2531

SEQ ID NO: 156

IIYDSASTYYASWAKG
CDR-H3 of DLX2531

SEQ ID NO: 157

ERAIFSGDFAL
CDR-L1 of DLX2531

SEQ ID NO: 158

QASQSIDNWLS
CDR-L2 of DLX2531

SEQ ID NO: 159

RASTLAS
CDR-L3 of DLX2531

SEQ ID NO: 160

QNTGGGVSIA
CDR-L1 of DLX2681

SEQ ID NO: 161

RASQSIGNWLS
CDR-L2 of DLX2681

SEQ ID NO: 162

RASNLAS
CDR-L3 of DLX2681

SEQ ID NO: 163

QNTGGGINIA

1. A method of treating autoimmune inner ear disease (AIED) in a subject in need thereof, comprising delivering to the subject a therapeutically affective amount of an antibody or fragment thereof that specifically binds to IL-1β, thereby treating AIED.

2. The method of claim 1, wherein the antibody or fragment thereof is administered via intratympanic injection.

3. The method of claim 1, wherein the antibody or fragment thereof is administered via parenteral administration.

4. The method of claim 1, wherein the antibody or fragment thereof is formulated into a pharmaceutical composition comprising a pharmaceutically acceptable carrier, diluent, or recipient.

5. The method of claim 1, wherein the antibody or fragment thereof is administered every 1, 2, 3, 4, 5, or 6 months.

6. (canceled)

7. The method of claim 1, wherein the antibody or fragment thereof is administered with at least one anti-inflammatory agent and/or immunosuppressive agent.

8. (canceled)

9. The method of claim 1, wherein the subject is human.

10. (canceled)

11. The method of claim 1, wherein the antibody or fragment thereof comprises:
a. variable heavy chain (VH) CDR sequences CDR-H1, CDR-H2 or CDR-1H3 as set forth in SEQ ID NOs:1, 2 and 3, respectively, and
b. variable light chain (VL) CDR sequences CDR-L1, CDR-L2 or CDR-L3 as set forth in SEQ ID NOs:4, 5, and 6, respectively.

12. The method of claim 1, wherein the antibody or fragment thereof has a potency (IC₅₀) with regard to inhibiting the biological effect of human IL-1β of lower than 50 pM as determined by inhibiting IL-1β stimulated release of IL-6 from human fibroblasts.

13. The method of claim 12, wherein the IC₅₀ is lower than about 30, 20, 10, 5, 4, 3, 2, or about 1 pM.

14. The method of claim 1, wherein the antibody or fragment thereof is a monovalent antibody fragment against IL-1β having a potency (IC₅₀) with regard to inhibiting the biological effect of human IL-1β of lower than 5 pM, as determined by inhibiting IL-1β stimulated release of IL-6 from human fibroblasts.

15. The method of claim 14, wherein the antibody or fragment thereof is a Fab, a Fab′, a scFv, a Fv fragment, a nanobody, a VHH or a minimal recognition unit.

16. The method of claim 1, wherein the antibody or fragment thereof is a full-length immunoglobulin or a bivalent antibody fragment.

17-19. (canceled)

20. The method of claim 1, wherein the antibody or fragment thereof comprises at least one light chain variable framework region FR-L1 of SEQ ID NO 18, the light chain variable framework region FR-L2 of SEQ ID NO: 19, the light chain variable framework region FR-L3 of SEQ ID NO:20 and/or the light chain variable framework region FR-L4 of SEQ ID NO:21.

21. The method of claim 1, wherein the antibody or fragment thereof comprises at least one heavy chain variable framework region FR-H1 of SEQ ID NOs:22, 26 or 30; the heavy chain variable framework region FR-H2 of SEQ ID NOs:23, 27 or 31; the heavy chain variable framework region FR-H3 of SEQ ID NOs:24, 28 or 32; and/or the heavy chain variable framework region FR-H4 of SEQ ID NOs:25, 29 or 33.

22. (canceled)

23. The method of claim 1, wherein the antibody or fragment thereof comprises:
a. a VH sequence selected from the group consisting of SEQ ID NO: 106, SEQ ID NO:107, SEQ ID NO:108, SEQ ID NO:109, SEQ ID NO:110, SEQ ID NO:111, SEQ ID NO: 112, SEQ ID NO:113, SEQ ID NO:114, SEQ ID NO:115, SEQ ID NO:116, SEQ ID NO:117, SEQ ID NO:118, SEQ ID NO:119, SEQ ID NO: 120, SEQ ID NO:121, SEQ ID NO:122, SEQ ID NO:124, SEQ ID NO:126, SEQ ID NO:128, SEQ ID NO:130, SEQ ID NO:132, SEQ ID NO: 134, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152; and/or
b. a VL sequence selected from the group consisting of SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 136, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151 and SEQ ID NO: 153.

24. The method of claim 1, wherein the antibody or fragment thereof comprises the linker sequence of SEQ ID NO: 9.

25. (canceled)

26. (canceled)

27. The method of claim 1, wherein the antibody or fragment thereof comprises a sequence selected from the group consisting of SEQ ID NOs:34 to 63, SEQ ID NO: 64, SEQ ID NO: 65, SEQ ID NO: 66, SEQ ID NO: 67, SEQ ID NO: 68 to 70, SEQ ID NO: 71 to 76, SEQ ID NO: 77 to 88, SEQ ID NO: 89 to 95 and SEQ ID NO: 154.

28. The method of claim 1, wherein the antibody or fragment thereof has the sequence SEQ ID NO: 10.

29. The method of claim 1, wherein the antibody or fragment thereof is humanized.

30. (canceled)


SEQUENCE LISTING (163 sequences)

SEQ ID NO: 1 - protein, 9 aa, Oryctolagus cuniculus
FSLSSAAMA

SEQ ID NO: 2 - protein, 16 aa, Oryctolagus cuniculus
IIYDSASTYYASWAKG

SEQ ID NO: 3 - protein, 11 aa, Oryctolagus cuniculus
ERAIFSGDFVL

SEQ ID NO: 4 - protein, 11 aa, Oryctolagus cuniculus
QASQSIDNWLS

SEQ ID NO: 5 - protein, 7 aa, Oryctolagus cuniculus
RASTLAS

SEQ ID NO: 6 - protein, 10 aa, Oryctolagus cuniculus
QNTGGGVSIA

SEQ ID NO: 7 - protein, 119 aa, Artificial Sequence
  humanized VH sequence
EVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 8 - protein, 109 aa, Artificial Sequence
  humanized VL sequence
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLG

SEQ ID NO: 9 - protein, 20 aa, Artificial Sequence
  linker sequence
GGGGSGGGGSGGGGSGGGGS

SEQ ID NO: 10 - protein, 248 aa, Artificial Sequence
  humanized IL-1 beta scFv
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 11 - protein, 9 aa, Artificial Sequence
  variant of CDR-H1
  misc_feature (5)..(6): Xaa can be any naturally occurring amino acid
FSLSXXAMA

SEQ ID NO: 12 - protein, 16 aa, Artificial Sequence
  Variant of CDR-H2
  misc_feature (3)..(4): Xaa can be any naturally occurring amino acid
  misc_feature (9)..(9): Xaa can be any naturally occurring amino acid
IIXXSASTXYASWAKG

SEQ ID NO: 13 - protein, 11 aa, Artificial Sequence
  variant of CDR-H3
  misc_feature (2)..(11): Xaa can be any naturally occurring amino acid
EXXXXXXXXXX

SEQ ID NO: 14 - protein, 11 aa, Artificial Sequence
  Variant of CDR-L1
  misc_feature (7)..(9): Xaa can be any naturally occurring amino acid
QASQSIXXXLS

SEQ ID NO: 15 - protein, 7 aa, Artificial Sequence
  Variant of CDR-L2
  misc_feature (1)..(1): Xaa can be any naturally occurring amino acid
  misc_feature (4)..(4): Xaa can be any naturally occurring amino acid
XASXLAS

SEQ ID NO: 16 - protein, 10 aa, Artificial Sequence
  Variant of CDR-L3
  misc_feature (3)..(3): Xaa can be any naturally occurring amino acid
  misc_feature (5)..(8): Xaa can be any naturally occurring amino acid
QNXGXXXXIA

SEQ ID NO: 17 - DNA, 744 bp, Artificial Sequence
  DNA sequence of DLX2323
gaaattgttatgacccagagcccgagcaccctgagcgcaagcgttggtgatcgtgtgattattacctgtcaggcaagccagagcattgataattggctgagctggtatcagcagaaaccgggtaaagcaccgaaactgctgatttatcgtgcaagcaccctggcaagcggtgttccgagccgttttagcggtagcggtagtggtgcagaatttaccctgaccattagcagcctgcagccggatgattttgcaacctattattgtcagaataccggtggtggtgttagcattgcatttggtcagggcaccaaactgaccgttctgggtggtggcggtggatccggtgggggtggtagcggaggtggtggttcaggcggtggtggcagcgaagttcagctggttgaaagtggtggtggtctggttcagcctggtggtagcctgcgtctgagctgtaccgcaagcggttttagcctgagcagcgcagcaatggcatgggttcgtcaggcacctggtaaaggtctggaatgggttggtattatctatgatagcgcaagcacctattatgcaagctgggcaaaaggtcgttttaccattagccgtgataccagtaaaaataccgtttacctgcagatgaatagtctgcgtgcagaggataccgcagtgtattattgtgcacgtgaacgtgcaattttcagcggtgattttgttctgtggggtcagggaaccctggttaccgttagcagc

SEQ ID NO: 18 - protein, 23 aa, Artificial Sequence
  framework sequence L1
EIVMTQSPSTLSASVGDRVIITC

SEQ ID NO: 19 - protein, 15 aa, Artificial Sequence
  framework sequence L2
WYQQKPGKAPKLLIY

SEQ ID NO: 20 - protein, 32 aa, Artificial Sequence
  framework sequence L3
GVPSRFSGSGSGAEFTLTISSLQPDDFATYYC

SEQ ID NO: 21 - protein, 11 aa, Artificial Sequence
  framework sequence L4
FGQGTKLTVLG

SEQ ID NO: 22 - protein, 26 aa, Artificial Sequence
  framework sequence H1
EVQLVESGGGLVQPGGSLRLSCTASG

SEQ ID NO: 23 - protein, 14 aa, Artificial Sequence
  framework sequence H2
WVRQAPGKGLEWVG

SEQ ID NO: 24 - protein, 32 aa, Artificial Sequence
  framework sequence H3
RFTISRDTSKNTVYLQMNSLRAEDTAVYYCAR

SEQ ID NO: 25 - protein, 11 aa, Artificial Sequence
  framework sequence H4
WGQGTLVTVSS

SEQ ID NO: 26 - protein, 26 aa, Artificial Sequence
  framework sequence H1
EVQLVESGGGLVQPGGSLRLSCTVSG

SEQ ID NO: 27 - protein, 14 aa, Artificial Sequence
  framework sequence H2
WVRQAPGKGLEWVG

SEQ ID NO: 28 - protein, 32 aa, Artificial Sequence
  framework sequence H3
RFTISKDTSKNTVYLQMNSLRAEDTAVYYCAR

SEQ ID NO: 29 - protein, 11 aa, Artificial Sequence
  framework sequence H4
WGQGTLVTVSS

SEQ ID NO: 30 - protein, 26 aa, Artificial Sequence
  framework sequence H1
EVQLVESGGGSVQPGGSLRLSCTASG

SEQ ID NO: 31 - protein, 14 aa, Artificial Sequence
  framework sequence H2
WVRQAPGKGLEWVG

SEQ ID NO: 32 - protein, 32 aa, Artificial Sequence
  framework sequence H3
RFTISRDTSKNTVYLQMNSLRAEDTASYYCAR

SEQ ID NO: 33 - protein, 11 aa, Artificial Sequence
  framework sequence H4
WGQGTTVTVSS

SEQ ID NO: 34 - protein, 248 aa, Artificial Sequence
  CDR-L1_D32X
  misc_feature (30)..(30): Xaa can be any naturally occurring amino acid
EIVMTQSPSTLSASVGDRVIITCQASQSIXNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 35 - protein, 248 aa, Artificial Sequence
  Variant CDR-L1_N33X
  misc_feature (31)..(31): Xaa can be any naturally occurring amino acid
EIVMTQSPSTLSASVGDRVIITCQASQSIDXWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 36 - protein, 248 aa, Artificial Sequence
  Variant CDR-L1_W40X
  misc_feature (32)..(32): Xaa can be any naturally occurring amino acid
EIVMTQSPSTLSASVGDRVIITCQASQSIDNXLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 37 - protein, 248 aa, Artificial Sequence
  Variant CDR-L2_R58X
  misc_feature (50)..(50): Xaa can be any naturally occurring amino acid
EIVMTQSPSTLSASVGDRVIITCQASQSIDNWLSWYQQKPGKAPKLLIYXASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 38 - protein, 248 aa, Artificial Sequence
  Variant CDR-L2_T69X
  misc_feature (53)..(53): Xaa can be any naturally occurring amino acid
EIVMTQSPSTLSASVGDRVIITCQASQSIDNWLSWYQQKPGKAPKLLIYRASXLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 39 - protein, 248 aa, Artificial Sequence
  Variant CDR-L3_T109X
  misc_feature (91)..(91): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNXGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 40 - protein, 248 aa, Artificial Sequence
  Variant CDR-L3_G111X
  misc_feature (93)..(93): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGXGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 41 - protein, 248 aa, Artificial Sequence
  Variant CDR-L3_G112X
  misc_feature (94)..(94): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGXVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 42 - protein, 248 aa, Artificial Sequence
  Variant CDR-L3_V135X
  misc_feature (95)..(95): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGXSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 43 - protein, 248 aa, Artificial Sequence
  Variant CDR-L3_S136X
  misc_feature (96)..(96): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVXIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 44 - protein, 248 aa, Artificial Sequence
  Variant CDR-H1_S33X
  misc_feature (160)..(160): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSXAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 45 - protein, 248 aa, Artificial Sequence
  Variant CDR-H1_A39X
  misc_feature (161)..(161): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSXAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 46 - protein, 248 aa, Artificial Sequence
  Variant CDR-H2_Y59X
  misc_feature (181)..(181): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIXDSASTYYASWAKGRFTISRDTSKNTVYLQMNSLRAEDTAVYYCARERAIFSGDFVLWGQGTLVTVSS

SEQ ID NO: 47 - protein, 248 aa, Artificial Sequence
  Variant CDR-H2_D60X
  misc_feature (182)..(182): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYXSASTYYASWAKGRFTISRDTSKNTVYLQMNSLRAEDTAVYYCARERAIFSGDFVLWGQGTLVTVSS

SEQ ID NO: 48 - protein, 248 aa, Artificial Sequence
  Variant CDR-H2_Y69X
  misc_feature (187)..(187): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTXYASWAKGRFTISRDTSKNTVYLQMNSLRAEDTAVYYCARERAIFSGDFVLWGQGTLVTVSS

SEQ ID NO: 49 - protein, 248 aa, Artificial Sequence
  Variant CDR-H3_R110X
  misc_feature (228)..(228): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCAREXAIFSGDFVLWGQGTLVTVSS

SEQ ID NO: 50 - protein, 248 aa, Artificial Sequence
  Variant CDR-H3_A111X
  misc_feature (229)..(229): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERXIFSGDFVLWGQGTLVTVSS

SEQ ID NO: 51 - protein, 248 aa, Artificial Sequence
  Variant CDR-H3_I112X
  misc_feature (230)..(230): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAXFSGDFVLWGQGTLVTVSS

SEQ ID NO: 52 - protein, 248 aa, Artificial Sequence
  Variant CDR-H3_F113X
  misc_feature (231)..(231): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIXSGDFVLWGQGTLVTVSS

SEQ ID NO: 53 - protein, 248 aa, Artificial Sequence
  Variant CDR-H3_S114X
  misc_feature (232)..(232): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFXGDFVLWGQGTLVTVSS

SEQ ID NO: 54 - protein, 248 aa, Artificial Sequence
  Variant CDR-H3_G115X
  misc_feature (233)..(233): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSXDFVLWGQGTLVTVSS

SEQ ID NO: 55 - protein, 248 aa, Artificial Sequence
  Variant CDR-H3_D135X
  misc_feature (234)..(234): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGXFVLWGQGTLVTVSS

SEQ ID NO: 56 - protein, 248 aa, Artificial Sequence
  Variant CDR-H3_F136X
  misc_feature (235)..(235): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDXVLWGQGTLVTVSS

SEQ ID NO: 57 - protein, 248 aa, Artificial Sequence
  Variant CDR-H3_V137X
  misc_feature (236)..(236): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFXLWGQGTLVTVSS

SEQ ID NO: 58 - protein, 248 aa, Artificial Sequence
  Variant CDR-H3_L138X
  misc_feature (237)..(237): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVXWGQGTLVTVSS

SEQ ID NO: 59 - protein, 248 aa, Artificial Sequence
  Variant DLX2464
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERQIFSGDMAGWGQGTLVTVSS

SEQ ID NO: 60 - protein, 248 aa, Artificial Sequence
  Variant DLX2465
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERNIFSGDMDLWGQGTLVTVSS

SEQ ID NO: 61 - protein, 248 aa, Artificial Sequence
  Variant DLX2466
EIVMTQSPSTLSASVGDRVIITCQASQSIGKYLSWYQQKPGKAPKLLIYRASTLASGVPSRFSGSGSGAEFTLTISSLQPDDFATYYCQNAGGGVSIAFGQGTKLTVLGGGGGSGGGGSGGGGSGGGGSEVQLVESGGGLVQPGGSLRLSCTASGFSLSSDAMAWVRQAPGKGLEWVGIIYDSASTYYASWAKGRFTISRDTSKNTVYLQMNSLRAEDTAVYYCARERNIFSGDMAGWGQGTLVTVSS

SEQ ID NO: 62 - protein, 248 aa, Artificial Sequence
  Variant DLX2467
EIVMTQSPSTLSASVGDRVIITCQASQSIHNWLSWYQQKPGKAPKLLIYRASNLASGVPSRFSGSGSGAEFTLTISSLQPDDFATYYCQNTGGGSSIAFGQGTKLTVLGGGGGSGGGGSGGGGSGGGGSEVQLVESGGGLVQPGGSLRLSCTASGFSLSRAAMAWVRQAPGKGLEWVGIIYDSASTYYASWAKGRFTISRDTSKNTVYLQMNSLRAEDTAVYYCARERMIFSGDFVLWGQGTLVTVSS

SEQ ID NO: 63 - protein, 248 aa, Artificial Sequence
  Variant DLX2468
EIVMTQSPSTLSASVGDRVIITCQASQSIGNYLSWYQQKPGKAPKLLIYRASTLASGVPSRFSGSGSGAEFTLTISSLQPDDFATYYCQNAGGGTSIAFGQGTKLTVLGGGGGSGGGGSGGGGSGGGGSEVQLVESGGGLVQPGGSLRLSCTASGFSLSSSAMAWVRQAPGKGLEWVGIIYDSASTYYASWAKGRFTISRDTSKNTVYLQMNSLRAEDTAVYYCARERNIFSGDMVLWGQGTLVTVSS

SEQ ID NO: 64 - protein, 248 aa, Artificial Sequence
  Variant DLX2475
EIVMTQSPSTLSASVGDRVIITCQASQSIDKWLSWYQQKPGKAPKLLIYQASTLASGVPSRFSGSGSGAEFTLTISSLQPDDFATYYCQNTGGGVHIAFGQGTKLTVLGGGGGSGGGGSGGGGSGGGGSEVQLVESGGGLVQPGGSLRLSCTASGFSLSSYAMAWVRQAPGKGLEWVGIIYDSASTYYASWAKGRFTISRDTSKNTVYLQMNSLRAEDTAVYYCARERAIFSGDFKLWGQGTLVTVSS

SEQ ID NO: 65 - protein, 248 aa, Artificial Sequence
  Variant DLX2476
EIVMTQSPSTLSASVGDRVIITCQASQSISSWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERDIFSGDFVGWGQGTLVTVSS

SEQ ID NO: 66 - protein, 248 aa, Artificial Sequence
  Variant DLX2480
EIVMTQSPSTLSASVGDRVIITCQASQSIDNWLSWYQQKPGKAPKLLIYRASTLASGVPSRFSGSGSGAEFTLTISSLQPDDFATYYCQNTGGGINIAFGQGTKLTVLGGGGGSGGGGSGGGGSGGGGSEVQLVESGGGLVQPGGSLRLSCTASGFSLSDAAMAWVRQAPGKGLEWVGIIYDSASTYYASWAKGRFTISRDTSKNTVYLQMNSLRAEDTAVYYCARERQIFSGDFVLWGQGTLVTVSS

SEQ ID NO: 67 - protein, 248 aa, Artificial Sequence
  Variant DLX2543
EIVMTQSPSTLSASVGDRVTITCQASQSISSWLSWYQQKPGKAPKLLIYKASTLASGVPSRFSGSGSGTEFTLTISSLQPDDFATYYCQNAGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 68 - protein, 248 aa, Artificial Sequence
  Variant DLX2529
EIVMTQSPSTLSASVGDRVIITCRASQSIGNWLSWYQQKPGKAPKLLIYRASNLASGVPSRFSGSGSGAEFTLTISSLQPEDFATYYCQNTGGGINIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 69 - protein, 249 aa, Artificial Sequence
  Variant DLX2547
ADIVMTQSPSTLSASVGDRVTITCQASQSISSYLSWYQQKPGKAPKLLIYRASTLASGVPSRFSGSGSGAEFTLTISSLQPDDFATYYCQNTGGGINIAFGQGTKLEIKRGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 70 - protein, 248 aa, Artificial Sequence
  Variant DLX2528
EIVMTQSPSTLSASVGDRVTITCQASQSIGNWLAWYQQKPGKAPKLLIYQASNLASGVPSRFSGSGSGTDFTLTISSLQPDDFATYYCQNAGGATTIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 71 - protein, 248 aa, Artificial Sequence
  Variant DLX2585
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTVSGFSLSSYAMSWVRQAPGKGLEWVGIIYDSASTYYASWAKGRFTISKDTSKNTVYLQMNSLRAEDTAVYYCARERAIFSGDFDYWGQGTLVTVSS

SEQ ID NO: 72 - protein, 248 aa, Artificial Sequence
  Variant DLX2545
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWIGIIYDSASTYYASWAKGRFTISRDTSKNTLYLQMNSLRAEDTAVYFCARERNIFSGDMVLWGQGTTVTVSS

SEQ ID NO: 73 - protein, 248 aa, Artificial Sequence
  Variant DLX2531
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGNVQPGGSLRLSCTASGFSLSNSAMAWVRQAPGKGLEWVGIIYDSASTYYASWAKGRFTISRDNSKNTVYLQMNSLRAEDTATYYCARERAIFSGDFALWGQGTLVTVSS

SEQ ID NO: 74 - protein, 248 aa, Artificial Sequence
  Variant DLX2586
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTVSGFSLSSYAMSWVRQAPGKGLEWIGIIYDSASTYYASWAKGRFTISKDTSKNTVYLQMNSLRAEDTAVYFCARERQIFSGDMDGWGQGTLVTVSS

SEQ ID NO: 75 - protein, 248 aa, Artificial Sequence
  Variant DLX2530
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGNVQPGGSLRLSCTASGFSLSDAAMAWVRQAPGKGLEWVGIIYDSASTFYASWAKGRFTISRDNSKNTLYLQMNSLRAEDTATYYCARERNIFSGDMALWGQGTTVTVSS

SEQ ID NO: 76 - protein, 248 aa, Artificial Sequence
  Variant DLX2548
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTVSGFSLSSYAMSWVRQAPGKGLEWIGIIYDSASTYYASWAKGRFTISKDTSKNTLYLQMNSLRAEDTAVYFCARERQIFSGDMDGWGQGTTVTVSS

SEQ ID NO: 77 - protein, 248 aa, Artificial Sequence
  Variant DLX2676
EIVMTQSPSTLSASVGDRVTITCQASQSIGNWLAWYQQKPGKAPKLLIYQASNLASGVPSRFSGSGSGTDFTLTISSLQPDDFATYYCQNAGGATTIAFGQGTKLTVLGGGGGSGGGGSGGGGSGGGGSEVQLVESGGGNVQPGGSLRLSCTASGFSLSDAAMAWVRQAPGKGLEWVGIIYDSASTFYASWAKGRFTISRDNSKNTLYLQMNSLRAEDTATYYCARERNIFSGDMALWGQGTTVTVSS

SEQ ID NO: 78 - protein, 248 aa, Artificial Sequence
  Variant DLX2677
EIVMTQSPSTLSASVGDRVTITCQASQSIGNWLAWYQQKPGKAPKLLIYQASNLASGVPSRFSGSGSGTDFTLTISSLQPDDFATYYCQNAGGATTIAFGQGTKLTVLGGGGGSGGGGSGGGGSGGGGSEVQLVESGGGNVQPGGSLRLSCTASGFSLSNSAMAWVRQAPGKGLEWVGIIYDSASTYYASWAKGRFTISRDNSKNTVYLQMNSLRAEDTATYYCARERAIFSGDFALWGQGTLVTVSS

SEQ ID NO: 79 - protein, 248 aa, Artificial Sequence
  Variant DLX2678
EIVMTQSPSTLSASVGDRVTITCQASQSIGNWLAWYQQKPGKAPKLLIYQASNLASGVPSRFSGSGSGTDFTLTISSLQPDDFATYYCQNAGGATTIAFGQGTKLTVLGGGGGSGGGGSGGGGSGGGGSEVQLVESGGGLVQPGGSLRLSCTVSGFSLSSYAMSWVRQAPGKGLEWIGIIYDSASTYYASWAKGRFTISKDTSKNTLYLQMNSLRAEDTAVYFCARERQIFSGDMDGWGQGTTVTVSS

SEQ ID NO: 80 - protein, 248 aa, Artificial Sequence
  Variant DLX2679
EIVMTQSPSTLSASVGDRVTITCQASQSIGNWLAWYQQKPGKAPKLLIYQASNLASGVPSRFSGSGSGTDFTLTISSLQPDDFATYYCQNAGGATTIAFGQGTKLTVLGGGGGSGGGGSGGGGSGGGGSEVQLVESGGGLVQPGGSLRLSCTVSGFSLSSYAMSWVRQAPGKGLEWVGIIYDSASTYYASWAKGRFTISKDTSKNTVYLQMNSLRAEDTAVYYCARERAIFSGDFDYWGQGTLVTVSS

SEQ ID NO: 81 - protein, 248 aa, Artificial Sequence
  Variant DLX2680
EIVMTQSPSTLSASVGDRVIITCRASQSIGNWLSWYQQKPGKAPKLLIYRASNLASGVPSRFSGSGSGAEFTLTISSLQPEDFATYYCQNTGGGINIAFGQGTKLTVLGGGGGSGGGGSGGGGSGGGGSEVQLVESGGGNVQPGGSLRLSCTASGFSLSDAAMAWVRQAPGKGLEWVGIIYDSASTFYASWAKGRFTISRDNSKNTLYLQMNSLRAEDTATYYCARERNIFSGDMALWGQGTTVTVSS

SEQ ID NO: 82 - protein, 248 aa, Artificial Sequence
  Variant DLX2681
EIVMTQSPSTLSASVGDRVIITCRASQSIGNWLSWYQQKPGKAPKLLIYRASNLASGVPSRFSGSGSGAEFTLTISSLQPEDFATYYCQNTGGGINIAFGQGTKLTVLGGGGGSGGGGSGGGGSGGGGSEVQLVESGGGNVQPGGSLRLSCTASGFSLSNSAMAWVRQAPGKGLEWVGIIYDSASTYYASWAKGRFTISRDNSKNTVYLQMNSLRAEDTATYYCARERAIFSGDFALWGQGTLVTVSS

SEQ ID NO: 83 - protein, 248 aa, Artificial Sequence
  Variant DLX2682
EIVMTQSPSTLSASVGDRVIITCRASQSIGNWLSWYQQKPGKAPKLLIYRASNLASGVPSRFSGSGSGAEFTLTISSLQPEDFATYYCQNTGGGINIAFGQGTKLTVLGGGGGSGGGGSGGGGSGGGGSEVQLVESGGGLVQPGGSLRLSCTVSGFSLSSYAMSWVRQAPGKGLEWIGIIYDSASTYYASWAKGRFTISKDTSKNTLYLQMNSLRAEDTAVYFCARERQIFSGDMDGWGQGTTVTVSS

SEQ ID NO: 84 - protein, 248 aa, Artificial Sequence
  Variant DLX2683
EIVMTQSPSTLSASVGDRVIITCRASQSIGNWLSWYQQKPGKAPKLLIYRASNLASGVPSRFSGSGSGAEFTLTISSLQPEDFATYYCQNTGGGINIAFGQGTKLTVLGGGGGSGGGGSGGGGSGGGGSEVQLVESGGGLVQPGGSLRLSCTVSGFSLSSYAMSWVRQAPGKGLEWVGIIYDSASTYYASWAKGRFTISKDTSKNTVYLQMNSLRAEDTAVYYCARERAIFSGDFDYWGQGTLVTVSS

SEQ ID NO: 85 - protein, 248 aa, Artificial Sequence
  Variant DLX2684
EIVMTQSPSTLSASVGDRVTITCQASQSISSWLSWYQQKPGKAPKLLIYKASTLASGVPSRFSGSGSGTEFTLTISSLQPDDFATYYCQNAGGGVSIAFGQGTKLTVLGGGGGSGGGGSGGGGSGGGGSEVQLVESGGGNVQPGGSLRLSCTASGFSLSDAAMAWVRQAPGKGLEWVGIIYDSASTFYASWAKGRFTISRDNSKNTLYLQMNSLRAEDTATYYCARERNIFSGDMALWGQGTTVTVSS

SEQ ID NO: 86 - protein, 248 aa, Artificial Sequence
  Variant DLX2685
EIVMTQSPSTLSASVGDRVTITCQASQSISSWLSWYQQKPGKAPKLLIYKASTLASGVPSRFSGSGSGTEFTLTISSLQPDDFATYYCQNAGGGVSIAFGQGTKLTVLGGGGGSGGGGSGGGGSGGGGSEVQLVESGGGNVQPGGSLRLSCTASGFSLSNSAMAWVRQAPGKGLEWVGIIYDSASTYYASWAKGRFTISRDNSKNTVYLQMNSLRAEDTATYYCARERAIFSGDFALWGQGTLVTVSS

SEQ ID NO: 87 - protein, 249 aa, Artificial Sequence
  Variant DLX2686
MEIVMTQSPSTLSASVGDRVTITCQASQSISSWLSWYQQKPGKAPKLLIYKASTLASGVPSRFSGSGSGTEFTLTISSLQPDDFATYYCQNAGGGVSIAFGQGTKLTVLGGGGGSGGGGSGGGGSGGGGSEVQLVESGGGLVQPGGSLRLSCTVSGFSLSSYAMSWVRQAPGKGLEWIGIIYDSASTYYASWAKGRFTISKDTSKNTLYLQMNSLRAEDTAVYFCARERQIFSGDMDGWGQGTTVTVSS

SEQ ID NO: 88 - protein, 248 aa, Artificial Sequence
  Variant DLX2687
EIVMTQSPSTLSASVGDRVTITCQASQSISSWLSWYQQKPGKAPKLLIYKASTLASGVPSRFSGSGSGTEFTLTISSLQPDDFATYYCQNAGGGVSIAFGQGTKLTVLGGGGGSGGGGSGGGGSGGGGSEVQLVESGGGLVQPGGSLRLSCTVSGFSLSSYAMSWVRQAPGKGLEWVGIIYDSASTYYASWAKGRFTISKDTSKNTVYLQMNSLRAEDTAVYYCARERAIFSGDFDYWGQGTLVTVSS

SEQ ID NO: 89 - protein, 249 aa, Artificial Sequence
  Variant DLX2689
ADIVMTQSPSTLSASVGDRVTITCQASQSISSYLSWYQQKPGKAPKLLIYKASTLASGVPSRFSGSGSGTDFTLTISSLQPEDFATYYCQNAGGGINIAFGQGTKVEIKRGGGGSGGGGSGGGGSGGGGSEVQLVESGGGNVQPGGSLRLSCTASGFSLSNSAMAWVRQAPGKGLEWVGIIYDSASTYYASWAKGRFTISRDNSKNTVYLQMNSLRAEDTATYYCARERAIFSGDFALWGQGTLVTVSS

SEQ ID NO: 90 - protein, 249 aa, Artificial Sequence
  Variant DLX2690
ADIVMTQSPSTLSASVGDRVTITCQASQSISSYLSWYQQKPGKAPKLLIYKASTLASGVPSRFSGSGSGTDFTLTISSLQPEDFATYYCQNAGGGINIAFGQGTKVEIKRGGGGSGGGGSGGGGSGGGGSEVQLVESGGGLVQPGGSLRLSCTVSGFSLSSYAMSWVRQAPGKGLEWIGIIYDSASTYYASWAKGRFTISKDTSKNTLYLQMNSLRAEDTAVYFCARERQIFSGDMDGWGQGTTVTVSS

SEQ ID NO: 91 - protein, 249 aa, Artificial Sequence
  Variant DLX2691
ADIVMTQSPSTLSASVGDRVTITCQASQSISSYLSWYQQKPGKAPKLLIYKASTLASGVPSRFSGSGSGTDFTLTISSLQPEDFATYYCQNAGGGINIAFGQGTKVEIKRGGGGSGGGGSGGGGSGGGGSEVQLVESGGGLVQPGGSLRLSCTVSGFSLSSYAMSWVRQAPGKGLEWVGIIYDSASTYYASWAKGRFTISKDTSKNTVYLQMNSLRAEDTAVYYCARERAIFSGDFDYWGQGTLVTVSS

SEQ ID NO: 92 - protein, 249 aa, Artificial Sequence
  Variant DLX2692
ADIVMTQSPSTLSASVGDRVTITCQASQSISSYLSWYQQKPGKAPKLLIYRASTLASGVPSRFSGSGSGAEFTLTISSLQPDDFATYYCQNTGGGINIAFGQGTKLEIKRGGGGSGGGGSGGGGSGGGGSEVQLVESGGGNVQPGGSLRLSCTASGFSLSDAAMAWVRQAPGKGLEWVGIIYDSASTFYASWAKGRFTISRDNSKNTLYLQMNSLRAEDTATYYCARERNIFSGDMALWGQGTTVTVSS

SEQ ID NO: 93 - protein, 249 aa, Artificial Sequence
  Variant DLX2693
ADIVMTQSPSTLSASVGDRVTITCQASQSISSYLSWYQQKPGKAPKLLIYRASTLASGVPSRFSGSGSGAEFTLTISSLQPDDFATYYCQNTGGGINIAFGQGTKLEIKRGGGGSGGGGSGGGGSGGGGSEVQLVESGGGNVQPGGSLRLSCTASGFSLSNSAMAWVRQAPGKGLEWVGIIYDSASTYYASWAKGRFTISRDNSKNTVYLQMNSLRAEDTATYYCARERAIFSGDFALWGQGTLVTVSS

SEQ ID NO: 94 - protein, 249 aa, Artificial Sequence
  Variant DLX2694
ADIVMTQSPSTLSASVGDRVTITCQASQSISSYLSWYQQKPGKAPKLLIYRASTLASGVPSRFSGSGSGAEFTLTISSLQPDDFATYYCQNTGGGINIAFGQGTKLEIKRGGGGSGGGGSGGGGSGGGGSEVQLVESGGGLVQPGGSLRLSCTVSGFSLSSYAMSWVRQAPGKGLEWIGIIYDSASTYYASWAKGRFTISKDTSKNTLYLQMNSLRAEDTAVYFCARERQIFSGDMDGWGQGTTVTVSS

SEQ ID NO: 95 - protein, 249 aa, Artificial Sequence
  Variant DLX2695
ADIVMTQSPSTLSASVGDRVTITCQASQSISSYLSWYQQKPGKAPKLLIYRASTLASGVPSRFSGSGSGAEFTLTISSLQPDDFATYYCQNTGGGINIAFGQGTKLEIKRGGGGSGGGGSGGGGSGGGGSEVQLVESGGGLVQPGGSLRLSCTVSGFSLSSYAMSWVRQAPGKGLEWVGIIYDSASTYYASWAKGRFTISKDTSKNTVYLQMNSLRAEDTAVYYCARERAIFSGDFDYWGQGTLVTVSS

SEQ ID NO: 96 - protein, 109 aa, Artificial Sequence
  VL CDR-L1_D32X
  misc_feature (30)..(30): Xaa can be any naturally occurring amino acid
EIVMTQSPSTLSASVGDRVIITCQASQSIXNWLSWYQQKPGKAPKLLIYRASTLASGVPSRFSGSGSGAEFTLTISSLQPDDFATYYCQNTGGGVSIAFGQGTKLTVLG

SEQ ID NO: 97 - protein, 109 aa, Artificial Sequence
  VL CDR-L1_N33X
  misc_feature (31)..(31): Xaa can be any naturally occurring amino acid
EIVMTQSPSTLSASVGDRVIITCQASQSIDXWLSWYQQKPGKAPKLLIYRASTLASGVPSRFSGSGSGAEFTLTISSLQPDDFATYYCQNTGGGVSIAFGQGTKLTVLG

SEQ ID NO: 98 - protein, 109 aa, Artificial Sequence
  VL CDR-L1_W40X
  misc_feature (32)..(32): Xaa can be any naturally occurring amino acid
EIVMTQSPSTLSASVGDRVIITCQASQSIDNXLSWYQQKPGKAPKLLIYRASTLASGVPSRFSGSGSGAEFTLTISSLQPDDFATYYCQNTGGGVSIAFGQGTKLTVLG

SEQ ID NO: 99 - protein, 109 aa, Artificial Sequence
  VL CDR-L2_R58X
  misc_feature (50)..(50): Xaa can be any naturally occurring amino acid
EIVMTQSPSTLSASVGDRVIITCQASQSIDNWLSWYQQKPGKAPKLLIYXASTLASGVPSRFSGSGSGAEFTLTISSLQPDDFATYYCQNTGGGVSIAFGQGTKLTVLG

SEQ ID NO: 100 - protein, 109 aa, Artificial Sequence
  VL CDR-L2_T69X
  misc_feature (53)..(53): Xaa can be any naturally occurring amino acid
EIVMTQSPSTLSASVGDRVIITCQASQSIDNWLSWYQQKPGKAPKLLIYRASXLASGVPSRFSGSGSGAEFTLTISSLQPDDFATYYCQNTGGGVSIAFGQGTKLTVLG

SEQ ID NO: 101 - protein, 109 aa, Artificial Sequence
  VL CDR-L3_T109X
  misc_feature (91)..(91): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNXGGGVSIAFGQGTKLTVLG

SEQ ID NO: 102 - protein, 109 aa, Artificial Sequence
  VL CDR-L3_G111X
  misc_feature (93)..(93): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGXGVSIAFGQGTKLTVLG

SEQ ID NO: 103 - protein, 109 aa, Artificial Sequence
  VL CDR-L3_G112X
  misc_feature (94)..(94): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGXVSIAFGQGTKLTVLG

SEQ ID NO: 104 - protein, 109 aa, Artificial Sequence
  VL CDR-L3_V135X
  misc_feature (95)..(95): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGXSIAFGQGTKLTVLG

SEQ ID NO: 105 - protein, 109 aa, Artificial Sequence
  VL CDR-L3_S136X
  misc_feature (96)..(96): Xaa can be any naturally occurring amino acid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVXIAFGQGTKLTVLG

SEQ ID NO: 106 - protein, 119 aa, Artificial Sequence
  VH CDR-H1_S33X
  misc_feature (31)..(31): Xaa can be any naturally occurring amino acid
EVQLVESGGGLVQPGGSLRLSCTASGFSLSXAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 107 - protein, 119 aa, Artificial Sequence
  VH CDR-H1_A39X
  misc_feature (32)..(32): Xaa can be any naturally occurring amino acid
EVQLVESGGGLVQPGGSLRLSCTASGFSLSSXAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 108 - protein, 119 aa, Artificial Sequence
  VH CDR-H2_Y59X
  misc_feature (52)..(52): Xaa can be any naturally occurring amino acid
EVQLVESGGGLVQPGGSLRLSCTASGFSLSSAAMAWVRQAPGKGLEWVGIIXDSASTYYASWAKGRFTISRDTSKNTVYLQMNSLRAEDTAVYYCARERAIFSGDFVLWGQGTLVTVSS

SEQ ID NO: 109 - protein, 119 aa, Artificial Sequence
  VH CDR-H2_D60X
  misc_feature (53)..(53): Xaa can be any naturally occurring amino acid
EVQLVESGGGLVQPGGSLRLSCTASGFSLSSAAMAWVRQAPGKGLEWVGIIYXSASTYYASWAKGRFTISRDTSKNTVYLQMNSLRAEDTAVYYCARERAIFSGDFVLWGQGTLVTVSS

SEQ ID NO: 110 - protein, 119 aa, Artificial Sequence
  VH CDR-H2_Y69X
  misc_feature (58)..(58): Xaa can be any naturally occurring amino acid
EVQLVESGGGLVQPGGSLRLSCTASGFSLSSAAMAWVRQAPGKGLEWVGIIYDSASTXYASWAKGRFTISRDTSKNTVYLQMNSLRAEDTAVYYCARERAIFSGDFVLWGQGTLVTVSS

SEQ ID NO: 111 - protein, 119 aa, Artificial Sequence
  VH CDR-H3_R110X
  misc_feature (99)..(99): Xaa can be any naturally occurring amino acid
EVQLVESGGGLVQPGGSLRLSCTASGFSLSSAAMAWVRQAPGKGLEWVGIIYDSASTYYASWAKGRFTISRDTSKNTVYLQMNSLRAEDTAVYYCAREXAIFSGDFVLWGQGTLVTVSS

SEQ ID NO: 112 - protein, 119 aa, Artificial Sequence
  VH CDR-H3_A111X
  misc_feature (100)..(100): Xaa can be any naturally occurring amino acid
EVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERXIFSGDFVLWGQGTLVTVSS

SEQ ID NO: 113 - protein, 119 aa, Artificial Sequence
  VH CDR-H3_I112X
  misc_feature (101)..(101): Xaa can be any naturally occurring amino acid
EVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAXFSGDFVLWGQGTLVTVSS

SEQ ID NO: 114 - protein, 119 aa, Artificial Sequence
  VH CDR-H3_F113X
  misc_feature (102)..(102): Xaa can be any naturally occurring amino acid
EVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIXSGDFVLWGQGTLVTVSS

SEQ ID NO: 115 - protein, 119 aa, Artificial Sequence
  VH CDR-H3_S114X
  misc_feature (103)..(103): Xaa can be any naturally occurring amino acid
EVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFXGDFVLWGQGTLVTVSS

SEQ ID NO: 116 - protein, 119 aa, Artificial Sequence
  VH CDR-H3_G115X
  misc_feature (104)..(104): Xaa can be any naturally occurring amino acid
EVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSXDFVLWGQGTLVTVSS

SEQ ID NO: 117 - protein, 119 aa, Artificial Sequence
  VH CDR-H3_D135X
  misc_feature (105)..(105): Xaa can be any naturally occurring amino acid
EVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGXFVLWGQGTLVTVSS

SEQ ID NO: 118 - protein, 119 aa, Artificial Sequence
  VH CDR-H3_F136X
  misc_feature (106)..(106): Xaa can be any naturally occurring amino acid
EVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDXVLWGQGTLVTVSS

SEQ ID NO: 119 - protein, 119 aa, Artificial Sequence
  VH CDR-H3_V137X
  misc_feature (107)..(107): Xaa can be any naturally occurring amino acid
EVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFXLWGQGTLVTVSS

SEQ ID NO: 120 - protein, 119 aa, Artificial Sequence
  VH CDR-H3_L138X
  misc_feature (108)..(108): Xaa can be any naturally occurring amino acid
EVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVXWGQGTLVTVSS

SEQ ID NO: 121 - protein, 119 aa, Artificial Sequence
  VH DLX2464
EVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERQIFSGDMAGWGQGTLVTVSS

SEQ ID NO: 122 - protein, 119 aa, Artificial Sequence
  VH DLX2465
EVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERNIFSGDMDLWGQGTLVTVSS

SEQ ID NO: 123 - protein, 109 aa, Artificial Sequence
  VL DLX2466
EIVMTQSPSTLSASVGDRVIITCQASQSIGKYLSWYQQKPGKAPKLLIYRASTLASGVPSRFSGSGSGAEFTLTISSLQPDDFATYYCQNAGGGVSIAFGQGTKLTVLG

SEQ ID NO: 124 - protein, 119 aa, Artificial Sequence
  VH DLX2466
EVQLVESGGGLVQPGGSLRLSCTASGFSLSSDAMAWVRQAPGKGLEWVGIIYDSASTYYASWAKGRFTISRDTSKNTVYLQMNSLRAEDTAVYYCARERNIFSGDMAGWGQGTLVTVSS

SEQ ID NO: 125 - protein, 109 aa, Artificial Sequence
  VL DLX2467
EIVMTQSPSTLSASVGDRVIITCQASQSIHNWLSWYQQKPGKAPKLLIYRASNLASGVPSRFSGSGSGAEFTLTISSLQPDDFATYYCQNTGGGSSIAFGQGTKLTVLG

SEQ ID NO: 126 - protein, 119 aa, Artificial Sequence
  VH DLX 2467
EVQLVESGGGLVQPGGSLRLSCTASGFSLSRAAMAWVRQAPGKGLEWVGIIYDSASTYYASWAKGRFTISRDTSKNTVYLQMNSLRAEDTAVYYCARERMIFSGDFVLWGQGTLVTVSS

SEQ ID NO: 127 - protein, 109 aa, Artificial Sequence
  VL DLX2468
EIVMTQSPSTLSASVGDRVIITCQASQSIGNYLSWYQQKPGKAPKLLIYRASTLASGVPSRFSGSGSGAEFTLTISSLQPDDFATYYCQNAGGGTSIAFGQGTKLTVLG

SEQ ID NO: 128 - protein, 119 aa, Artificial Sequence
  VH DLX2468
EVQLVESGGGLVQPGGSLRLSCTASGFSLSSSAMAWVRQAPGKGLEWVGIIYDSASTYYASWAKGRFTISRDTSKNTVYLQMNSLRAEDTAVYYCARERNIFSGDMVLWGQGTLVTVSS

SEQ ID NO: 129 - protein, 109 aa, Artificial Sequence
  VL DLX2475
EIVMTQSPSTLSASVGDRVIITCQASQSIDKWLSWYQQKPGKAPKLLIYQASTLASGVPSRFSGSGSGAEFTLTISSLQPDDFATYYCQNTGGGVHIAFGQGTKLTVLG

SEQ ID NO: 130 - protein, 119 aa, Artificial Sequence
  VH DLX2475
EVQLVESGGGLVQPGGSLRLSCTASGFSLSSYAMAWVRQAPGKGLEWVGIIYDSASTYYASWAKGRFTISRDTSKNTVYLQMNSLRAEDTAVYYCARERAIFSGDFKLWGQGTLVTVSS

SEQ ID NO: 131 - protein, 109 aa, Artificial Sequence
  VL DLX2476
EIVMTQSPSTLSASVGDRVIITCQASQSISSWLSWYQQKPGKAPKLLIYRASTLASGVPSRFSGSGSGAEFTLTISSLQPDDFATYYCQNTGGGVSIAFGQGTKLTVLG

SEQ ID NO: 132 - protein, 119 aa, Artificial Sequence
  VH DLX2476
EVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERDIFSGDFVGWGQGTLVTVSS

SEQ ID NO: 133 - protein, 109 aa, Artificial Sequence
  VL DLX2480
EIVMTQSPSTLSASVGDRVIITCQASQSIDNWLSWYQQKPGKAPKLLIYRASTLASGVPSRFSGSGSGAEFTLTISSLQPDDFATYYCQNTGGGINIAFGQGTKLTVLG

SEQ ID NO: 134 - protein, 119 aa, Artificial Sequence
  VH DLX2480
EVQLVESGGGLVQPGGSLRLSCTASGFSLSDAAMAWVRQAPGKGLEWVGIIYDSASTYYASWAKGRFTISRDTSKNTVYLQMNSLRAEDTAVYYCARERQIFSGDFVLWGQGTLVTVSS

SEQ ID NO: 135 - protein, 109 aa, Artificial Sequence
  VL DLX2543
EIVMTQSPSTLSASVGDRVTITCQASQSISSWLSWYQQKPGKAPKLLIYKASTLASGVPSRFSGSGSGTEFTLTISSLQPDDFATYYCQNAGGGVSIAFGQGTKLTVLG

SEQ ID NO: 136 - protein, 109 aa, Artificial Sequence
  VL DLX2529
EIVMTQSPSTLSASVGDRVIITCRASQSIGNWLSWYQQKPGKAPKLLIYRASNLASGVPSRFSGSGSGAEFTLTISSLQPEDFATYYCQNTGGGINIAFGQGTKLTVLG

SEQ ID NO: 137 - protein, 110 aa, Artificial Sequence
  VL DLX2547
ADIVMTQSPSTLSASVGDRVTITCQASQSISSYLSWYQQKPGKAPKLLIYRASTLASGVPSRFSGSGSGAEFTLTISSLQPDDFATYYCQNTGGGINIAFGQGTKLEIKR

SEQ ID NO: 138 - protein, 119 aa, Artificial Sequence; identical to SEQ ID NO: 7
  VH DLX2547

SEQ ID NO: 139 - protein, 109 aa, Artificial Sequence
  VL DLX2528
EIVMTQSPSTLSASVGDRVTITCQASQSIGNWLAWYQQKPGKAPKLLIYQASNLASGVPSRFSGSGSGTDFTLTISSLQPDDFATYYCQNAGGATTIAFGQGTKLTVLG

SEQ ID NO: 140 - protein, 119 aa, Artificial Sequence; identical to SEQ ID NO: 7
  VH DLX2528

SEQ ID NO: 141 - protein, 109 aa, Artificial Sequence; identical to SEQ ID NO: 8
  VL DLX2585

SEQ ID NO: 142 - protein, 119 aa, Artificial Sequence
  VH DLX2585
EVQLVESGGGLVQPGGSLRLSCTVSGFSLSSYAMSWVRQAPGKGLEWVGIIYDSASTYYASWAKGRFTISKDTSKNTVYLQMNSLRAEDTAVYYCARERAIFSGDFDYWGQGTLVTVSS

SEQ ID NO: 143 - protein, 109 aa, Artificial Sequence; identical to SEQ ID NO: 8
  VL DLX2545

SEQ ID NO: 144 - protein, 119 aa, Artificial Sequence
  VH DLX2545
EVQLVESGGGLVQPGGSLRLSCTASGFSLSSAAMAWVRQAPGKGLEWIGIIYDSASTYYASWAKGRFTISRDTSKNTLYLQMNSLRAEDTAVYFCARERNIFSGDMVLWGQGTTVTVSS

SEQ ID NO: 145 - protein, 109 aa, Artificial Sequence; identical to SEQ ID NO: 8
  VL DLX2531

SEQ ID NO: 146 - protein, 119 aa, Artificial Sequence
  VH DLX2531
EVQLVESGGGNVQPGGSLRLSCTASGFSLSNSAMAWVRQAPGKGLEWVGIIYDSASTYYASWAKGRFTISRDNSKNTVYLQMNSLRAEDTATYYCARERAIFSGDFALWGQGTLVTVSS

SEQ ID NO: 147 - protein, 109 aa, Artificial Sequence; identical to SEQ ID NO: 8
  VL DLX2586

SEQ ID NO: 148 - protein, 119 aa, Artificial Sequence
  VH DLX2586
EVQLVESGGGLVQPGGSLRLSCTVSGFSLSSYAMSWVRQAPGKGLEWIGIIYDSASTYYASWAKGRFTISKDTSKNTVYLQMNSLRAEDTAVYFCARERQIFSGDMDGWGQGTLVTVSS

SEQ ID NO: 149 - protein, 109 aa, Artificial Sequence; identical to SEQ ID NO: 8
  VL DLX2530

SEQ ID NO: 150 - protein, 119 aa, Artificial Sequence
  VH DLX2530
EVQLVESGGGNVQPGGSLRLSCTASGFSLSDAAMAWVRQAPGKGLEWVGIIYDSASTFYASWAKGRFTISRDNSKNTLYLQMNSLRAEDTATYYCARERNIFSGDMALWGQGTTVTVSS

SEQ ID NO: 151 - protein, 109 aa, Artificial Sequence; identical to SEQ ID NO: 8
  VL DLX2548

SEQ ID NO: 152 - protein, 119 aa, Artificial Sequence
  VH DLX2548
EVQLVESGGGLVQPGGSLRLSCTVSGFSLSSYAMSWVRQAPGKGLEWIGIIYDSASTYYASWAKGRFTISKDTSKNTLYLQMNSLRAEDTAVYFCARERQIFSGDMDGWGQGTTVTVSS

SEQ ID NO: 153 - protein, 110 aa, Artificial Sequence
  VL DLX2544
ADIVMTQSPSTLSASVGDRVTITCQASQSISSYLSWYQQKPGKAPKLLIYKASTLASGVPSRFSGSGSGTDFTLTISSLQPEDFATYYCQNAGGGINIAFGQGTKVEIKR

SEQ ID NO: 154 - protein, 249 aa, Artificial Sequence
  DLX2544
ADIVMTQSPSTLSASVGDRVTITCQASQSISSYLSWYQQKPGKAPKLLIYKASTLASGVPSRFSGSGSGTDFTLTISSLQPEDFATYYCQNAGGGINIAFGQGTKVEIKRGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSS

SEQ ID NO: 155 - protein, 9 aa, Artificial Sequence
  CDR-H1 of DLX2531
FSLSNSAMA

SEQ ID NO: 156 - protein, 16 aa, Artificial Sequence
  CDR-H2 of DLX2531
IIYDSASTYYASWAKG

SEQ ID NO: 157 - protein, 11 aa, Artificial Sequence
  CDR-H3 of DLX2531
ERAIFSGDFAL

SEQ ID NO: 158 - protein, 11 aa, Artificial Sequence
  CDR-L1 of DLX2531
QASQSIDNWLS

SEQ ID NO: 159 - protein, 7 aa, Artificial Sequence
  CDR-L2 of DLX2531
RASTLAS

SEQ ID NO: 160 - protein, 10 aa, Artificial Sequence
  CDR-L3 of DLX2531
QNTGGGVSIA

SEQ ID NO: 161 - protein, 11 aa, Artificial Sequence
  CDR-L1 of DLX2681
RASQSIGNWLS

SEQ ID NO: 162 - protein, 7 aa, Artificial Sequence
  CDR-L2 of DLX2681
RASNLAS

SEQ ID NO: 163 - protein, 10 aa, Artificial Sequence
  CDR-L3 of DLX2681
QNTGGGINIA